(19) United States
(12) Patent Application Publication
(10) Pub. No.: US 2023/0173073 A1
(43) Pub. Date: Jun. 8, 2023

(54) CO-GROUND ACTIVE(S) COMPRISING PRODUCT COMPRISING SURFACE-REACTED CALCIUM CARBONATE

(71) Applicant: Omya International AG, Oftringen (CH)

(72) Inventors: Florentine Marianne HILTY-VANCURA, Meilen (CH); Laura DE MIGUEL, Zofingen (CH)

(73) Assignee: Omya International AG, Oftringen (CH)

(21) Appl. No.: 17/998,050

(22) PCT Filed: May 19, 2021
(86) PCT No.: PCT/EP2021/063374

§ 371 Date: Nov. 7, 2022

(30) Foreign Application Priority Data
Jun. 25, 2020 (EP) 20182332.5

(51) Int. Cl. A61K 47/12; A61K 9/14

(52) U.S. Cl. CPC A61K 47/12; A61K 9/145

(57) ABSTRACT

The present invention refers to a Co-ground active(s) comprising product comprising a carrier material being surface-reacted calcium carbonate, a method for preparing said co-ground active(s) comprising product, a pharmaceutical, nutraceutical, veterinary or agricultural product comprising the co-ground active(s) comprising product according as well as the use of the surface-reacted calcium carbonate as a carrier material for reducing the X-ray crystallinity of solid active(s).

The present invention relates to a co-ground active(s) comprising product comprising a carrier material being surface-reacted calcium carbonate, a method for preparing said co-ground active(s) comprising product, a pharmaceutical, nutraceutical, veterinary or agricultural product comprising the co-ground active(s) comprising product according as well as the use of the surface-reacted calcium carbonate as a carrier material for reducing the X-ray crystallinity of solid active(s).

In many applications such as pharmaceutical, nutraceutical, veterinary and agricultural products solid active ingredients (also named “actives”) are added. However, many of these actives have poor water and/or acid solubility which often adversely affects their bioavailability. For example, several pharmaceutical actives typically have low acid solubility and thus show low absorption from the gastro intestinal tract. Other actives such as nutraceutical and agricultural actives typically have low water solubility resulting in a low absorption into the respective body.

In view of this, several methods have been developed for increasing the solubility of poorly soluble actives: encapsulation (cyclodextrines), solid dispersions with polymers (often by hot-melt extrusion), or the use of wetting/surfacting agents. Furthermore, it is well known that the amorphous forms of actives show a better solubility and dissolution rate than their crystalline forms. Thus, methods have been developed for amorphizing of actives such that they reach higher solubility and dissolution rates resulting in a higher bioavailability for e.g. pharmaceutical and neutraceutical actives. A further advantage of the amorphization of actives is that less of the amorphized active can be used in order to reach similar plasma levels compared to the same crystalline active. In this regard, amorphization by co-milling with excipients such as PVP (polyvinylpirrolidone), MCC (microcrystalline cellulose), cyclodextrins as well as with porous carriers such as mesoporous silica particles and silicates have been proposed.

For example, US20140206717 A1 refers to a method for producing a substantially amorphous stable drug product comprising preparing an amorphous dispersion of an active pharmaceutical ingredients in the presence of an inorganic matrix, e.g. magnesium aluminometasilicate, and a secondary polymer. US20130095177 A1 refers to a method of preparing an intermediate comprising fingolimod and one or more pharmaceutically acceptable excipients, comprising the steps of: (i) optionally mixing (a) fingolimod and (b) the excipient or the plurality of excipients, (ii) jointly comminuting (a) fingolimod and (b) the one or more excipients into intermediate particles such that 90 per cent by volume of all the resulting intermediate particles have a particle size of less than 250 µm and greater than 0.6 µm. WO9917736 A1 refers to co-grounds for the cosmetic and dermatological use, obtainable by co-grinding, without the use of solvents, at least two solid components, in the homogeneously split crystalline or amorphous forms selected from cosmetic actives, carriers to modify the hydrophilic or lipophilic characteristics of the system or its release or absorption characteristics, and/or inert inorganic materials.

However, the products described are typically prepared by using high energy input for achieving a high amorphization and thus the preparation of amorphized actives comprising products is highly energy and cost consuming.

Thus, there is still a need in the art for carrier materials allowing an efficient amorphization of actives. It is further desired that the amorphized actives comprising product is obtained by a sufficient energy input. Furthermore, it is desired that the carrier material allows a sufficient loading with actives.

One or more of the foregoing and other objects are solved by the subject-matter as defined herein in the independent claims. Advantageous embodiments of the present invention are defined in the corresponding sub-claims.

The present invention thus relates to a co-ground active(s) comprising product comprising a carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment, and one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding

According to one embodiment, the one or more at least partially X-ray amorphous active(s) has/have a crystallinity of less than 50 wt.-%, preferably of less than 40 wt.-%, more preferably of less than 30 wt.-% and most preferably of less than 20 wt.-%, based on the total weight of the one or more at least partially X-ray amorphous active.

According to another embodiment, the natural ground calcium carbonate is selected from calcium carbonate containing minerals selected from the group comprising marble, chalk, limestone and mixtures thereof.

According to yet another embodiment, the surface-reacted calcium carbonate is a reaction product of natural ground calcium carbonate with carbon dioxide and phosphoric acid, wherein the carbon dioxide is formed in-situ by the phosphoric acid treatment.

According to one embodiment, the one or more at least partially X-ray amorphous active(s) has/have a melting point of at least 30° C., more preferably at least 35° C. and most preferably in the range from 35 to 400° C.

According to another embodiment, the co-ground active(s) product comprises the one or more at least partially X-ray amorphous active(s) in an amount ranging from 1 to 45 wt.-%, preferably from 2 to 35 wt.-% and most preferably from 3 to 30 wt.-%, based on the total weight of the co-ground active(s) comprising product.

According to yet another embodiment, the carrier material of the co-ground active(s) comprising product is free of materials differing from surface-reacted calcium carbonate.

According to a further aspect, a method for preparing the co-ground active(s) comprising product is provided, the method comprising the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,
wherein the co-grinding in step c) is carried out in the absence of solvent(s).

According to one embodiment, the surface-reacted calcium carbonate provided in step a) a) has a BET specific surface area of from 1 m²/g to 200 m²/g, preferably 2 m²/g to 150 m²/g, more preferably 20 m²/g to 140 m²/g, most preferably 40 m²/g to 110 m²/g, measured using nitrogen and the BET method according to ISO 9277:2010; and/or, b) comprises particles having a volume median grain diameter d₅₀(vol) of from 0.5 to 50 µm, preferably from 0.7 to 25 µm, more preferably 0.8 to 20 µm, particularly 1 to 10 µm as measured by laser diffraction; and/or c) has an intra-particle intruded specific pore volume within the range of 0.15 to 1.60 cm³/g, preferably from 0.30 to 1.50 cm³/g, more preferably from 0.30 to 1.40 cm³/g, and most preferably from 0.30 to 1.35 cm³/g, calculated from a mercury intrusion porosimetry measurement.

According to another embodiment, the co-grinding in step c) is carried out in a mill, preferably selected from a ball mill, such as a planetary ball mill, roller mill, table mill, sand mill, ring roller mill, rod mill, vibrating mill, centrifugal impact mill, vertical bead mill and attrition mill.

According to one embodiment, the co-grinding in step c) is carried out at an energy input of at least 100 kJ/kg, preferably at least 150 kJ/kg, more preferably at least 200 kJ/kg and most preferably in the range from 200 to 1300 kJ/kg.

According to another aspect, a pharmaceutical, nutraceutical, veterinary or agricultural product comprising the co-ground active(s) comprising product is provided.

According to one embodiment, the pharmaceutical, nutraceutical, veterinary or agricultural product is in the form of a liquid dosage form, preferably a liquid dosage form such as an emulsion, dispersion, creme, solution, spray or inhalation spray, or solid dosage form, preferably a solid dosage form such as a powder, tablet, mini-tablet, sachet, granule, capsule, suppositories, or film.

According to another aspect, the use of a surface-reacted calcium carbonate as a carrier material for reducing the X-ray crystallinity of solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof is provided, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment.

It should be understood that for the purpose of the present invention the following terms have the following meaning.

The term “surface-reacted” in the meaning of the present application shall be used to indicate that a natural ground calcium carbonate has been subjected to a process comprising at least a partial, i.e. a partialor (substantially) complete, dissolution of said natural ground calcium carbonate upon treatment with with one or more H₃O⁺ ion donors (e.g., by use of water-soluble free acids and/or acidic salts) in an aqueous environment followed by a crystallization process which may occur in the absence or presence of further crystallization additive(s).

An “H₃O⁺ ion donor” in the context of the present invention is a Brønsted acid and/or an acid salt, i.e. a salt containing an acidic hydrogen.

The term “acid” as used herein refers to an acid in the meaning of the definition by Brønsted and Lowry (e.g., H₃PO₄, H₂PO₄⁻, HPO₄²⁻).

In the meaning of the present invention “water-insoluble” materials are defined as materials which, when mixed with deionised water and filtered on a filter having a 0.2 µm pore size at 20° C. to recover the liquid filtrate, provide less than or equal to 0.1 g of recovered solid material following evaporation at 95 to 100° C. of 100 g of said liquid filtrate. “Water-soluble” materials are defined as materials leading to the recovery of greater than 0.1 g of recovered solid material following evaporation at 95 to 100° C. of 100 g of said liquid filtrate.

“Natural ground calcium carbonate” (GCC) in the meaning of the present invention is a calcium carbonate obtained from natural sources, such as limestone, marble, or chalk, and processed through a wet and/or dry treatment such as grinding, screening and/or fractionating, for example, by a cyclone or classifier.

The BET specific surface area in the meaning of the present invention is defined as the surface area of the particles divided by the mass of the particles. As used therein the specific surface area is measured by adsorption using the BET isotherm (ISO 9277:2010) and is specified in m²/g.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

Terms like “obtainable” or “definable” and “obtained” or “defined” are used interchangeably. This e.g. means that, unless the context clearly dictates otherwise, the term “obtained” does not mean to indicate that e.g. an embodiment must be obtained by e.g. the sequence of steps following the term “obtained” though such a limited understanding is always included by the terms “obtained” or “defined” as a preferred embodiment.

According to the present invention, the co-ground active(s) comprising product comprises a carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment, and one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding.

It has been especially found out that the co-ground active(s) comprising product comprises a carrier material allowing an efficient amorphization of actives and a sufficient loading with such actives. Furthermore, the amorphized actives comprising product is obtained by a sufficient energy input.

In the following, it is referred to further details of the present invention and especially the foregoing co-ground active(s) comprising product.

One requirement of the present invention is that the co-ground active(s) comprising product is obtained by co-grinding, without the use of solvents. That is to say, the carrier material being surface-reacted calcium carbonate and one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof are co-ground without the use of solvents.

In general, only the carrier material being surface-reacted calcium carbonate and the one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof are co-ground without the use of solvents. In this embodiment, the co-ground active(s) comprising product thus consists of the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment, and the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding.

However, if it is desired to modify the characteristics, such as the chemical or sensory characteristics, of the co-ground active(s) comprising product, it may be also possible to co-grind the carrier material being surface-reacted calcium carbonate and the one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof in the presence of additive(s) without the use of solvents.

However, such additive(s) is/are present in minor amounts and is/are preferably present in amounts of less than 15 wt.-%, more preferably less than 10 wt.-% and most preferably less than 5 wt.-%, based on the total weight of the co-ground active(s) comprising product. If present, such additive(s) is/are preferably present in amounts of more than 0.001 wt.-%, based on the total weight of the co-ground active(s) comprising product.

Thus, the co-ground active(s) comprising product may consist of the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment, the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof agricultural active(s) and/or inactive precursor thereof, and mixtures thereof, wherein the one or more active(s) is/are rendered at least partially X-ray amorphous during co-grinding, and additive(s) in an amount of less than 15 wt.-%, more preferably less than 10 wt.-% and most preferably less than 5 wt.-%, based on the total weight of the co-ground active(s) comprising product.

It is appreciated that the weight ratio of the carrier material to the one or more at least partially X-ray amorphous active(s) may vary in wide limits in the co-ground active(s) comprising product. For example, the weight ratio of the carrier material to the one or more at least partially X-ray amorphous active(s) 99:1 to about 55:1.

Thus, it is preferred that the co-ground active(s) comprising product comprises the one or more at least partially X-ray amorphous active(s) in an amount ranging from 1 to 45 wt.-%, preferably from 2 to 35 wt.-% and most preferably from 3 to 30 wt.-%, based on the total weight of the co-ground active(s) comprising product.

Hence, the co-ground active(s) comprising product preferably comprises, more preferably consists of,
i) the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment, and
ii) from 1 to 45 wt.-%, preferably from 2 to 35 wt.-% and most preferably from 3 to 30 wt.-%, based on the total weight of the co-ground active(s) comprising product, of the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding.

For example, the co-ground active(s) comprising product preferably comprises, more preferably consists of,
i) from 55 to 99 wt.-%, preferably from 65 to 98 wt.-% and most preferably from 70 to 97 wt.-%, based on the total weight of the co-ground active(s) comprising product, of the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment, and
ii) from 1 to 45 wt.-%, preferably from 2 to 35 wt.-% and most preferably from 3 to 30 wt.-%, based on the total weight of the co-ground active(s) comprising product, of the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding.

If the co-ground active(s) comprising product further comprises additive(s), the co-ground active(s) comprising product preferably comprises, more preferably consists of,
i) the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the one or more H₃O⁺ ion donorstreatment,
ii) from 1 to 45 wt.-%, preferably from 2 to 35 wt.-% and most preferably from 3 to 30 wt.-%, based on the total weight of the co-ground active(s) comprising product, of the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding, and
iii) less than 15 wt.-%, more preferably less than 10 wt.-% and most preferably less than 5 wt.-%, based on the total weight of the co-ground active(s) comprising product, of the additive(s).

For example, the co-ground active(s) comprising product preferably comprises, more preferably consists of,
i) the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the one or more H₃O⁺ ion donorstreatment,
ii) from 1 to 45 wt.-%, preferably from 2 to 35 wt.-% and most preferably from 3 to 30 wt.-%, based on the total weight of the co-ground active(s) comprising product, of the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding, and
iii) from 0.001 to 15 wt.-%, more preferably from 0.001 to 10 wt.-% and most preferably from 0.001 to 5 wt.-%, based on the total weight of the co-ground active(s) comprising product, of the additive(s).

If the co-ground active(s) comprising product further comprises additive(s), the co-ground active(s) comprising product preferably comprises, more preferably consists of,
i) from 40 to 99 wt.-%, preferably from 55 to 98 wt.-% and most preferably from 65 to 97 wt.-%, based on the total weight of the co-ground active(s) comprising product, of the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment,
ii) from 1 to 45 wt.-%, preferably from 2 to 35 wt.-% and most preferably from 3 to 30 wt.-%, based on the total weight of the co-ground active(s) comprising product, of the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding, and
iii) less than 15 wt.-%, more preferably less than 10 wt.-% and most preferably less than 5 wt.-%, based on the total weight of the co-ground active(s) comprising product, of the additive(s).

For example, the co-ground active(s) comprising product preferably comprises, more preferably consists of,
i) from 40 to 98.999 wt.-%, preferably from 55 to 97.999 wt.-% and most preferably from 65 to 96.999 wt.-%, based on the total weight of the co-ground active(s) comprising product, of the carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment,
ii) from 1 to 45 wt.-%, preferably from 2 to 35 wt.-% and most preferably from 3 to 30 wt.-%, based on the total weight of the co-ground active(s) comprising product, of the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding, and
iii) from 0.001 to 15 wt.-%, more preferably from 0.001 to 10 wt.-% and most preferably from 0.001 to 5 wt.-%, based on the total weight of the co-ground active(s) comprising product, of the additive(s).

One requirement of the present invention is that the co-ground active(s) comprising product comprises a carrier material being surface-reacted calcium carbonate. The surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment. In a preferred embodiment, the surface-reacted calcium carbonate is obtained by a process comprising the steps of: (a) providing a suspension of natural ground calcium carbonate, (b) adding at least one acid having a pKₐ value of 0 or less at 20° C. or having a pKₐ value from 0 to 2.5 at 20° C. to the suspension of step a), and (c) treating the suspension of step (a) with carbon dioxide before, during or after step (b). According to another embodiment the surface-reacted calcium carbonate is obtained by a process comprising the steps of: (A) providing a natural ground calcium carbonate, (B) providing at least one water-soluble acid, (C) providing gaseous CO₂, (D) contacting said natural ground calcium carbonate of step (A) with the at least one acid of step (B) and with the CO₂ of step (C), characterised in that: (i) the at least one acid of step B) has a pKₐ of greater than 2.5 and less than or equal to 7 at 20° C., associated with the ionisation of its first available hydrogen, and a corresponding anion is formed on loss of this first available hydrogen capable of forming a water-soluble calcium salt, and (ii) following contacting the at least one acid with natural ground calcium carbonate, at least one water-soluble salt, which in the case of a hydrogen-containing salt has a pKₐ of greater than 7 at 20° C., associated with the ionisation of the first available hydrogen, and the salt anion of which is capable of forming water-insoluble calcium salts, is additionally provided.

In a preferred embodiment of the invention, the surface-reacted calcium carbonate is obtained by a process comprising the steps of: (a) providing a suspension of natural ground calcium carbonate, (b) adding phosphoric acid to the suspension of step a), and (c) treating the suspension of step (a) with carbon dioxide before, during or after step (b).

“Natural ground calcium carbonate” (GCC) preferably is selected from calcium carbonate containing minerals selected from the group comprising marble, chalk, limestone and mixtures thereof.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

It is to be noted that surface-reacted calcium carbonate may be also prepared by using precipitated calcium carbonate instead of natural ground calcium carbonate. In this case, the surface-reacted calcium carbonate is a reaction product of precipitated calcium carbonate with carbon dioxide and one or more H₃O⁺ ion donors, wherein the carbon dioxide is formed in-situ by the H₃O⁺ ion donors treatment. “Precipitated calcium carbonate” (PCC) in the meaning of the present invention is a synthesized material, generally obtained by precipitation following reaction of carbon dioxide and calcium hydroxide in an aqueous environment or by precipitation of calcium and carbonate ions, for example CaCl₂ and Na₂CO₃, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the natural ground calcium carbonate is in form of particles having a weight median particle size d₅₀(wt) of 0.05 to 10.0 µm, preferably 0.2 to 5.0 µm, more preferably 0.4 to 3.0 µm, most preferably 0.6 to 1.2 µm, especially 0.7 µm. According to a further embodiment of the present invention, the natural ground calcium carbonate is in form of particles having a top cut particle size d₉₈(wt) of 0.15 to 55 µm, preferably 1 to 40 µm, more preferably 2 to 25 µm, most preferably 3 to 15 µm, especially 4 µm.

The natural ground calcium carbonate may be used dry or suspended in water. Preferably, a corresponding slurry has a content of natural ground calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H₃O⁺ ion donors used for the preparation of surface-reacted calcium carbonate may be any strong acid, medium-strong acid, or weak acid, or mixtures thereof, generating H₃O⁺ ions under the preparation conditions. According to the present invention, the at least one H₃O⁺ ion donor can also be an acid salt, generating H₃O⁺ ions under the preparation conditions.

According to one embodiment, the one or more H₃O⁺ ion donors is/are a strong acid having a pKₐ of 0 or less at 20° C.

According to another embodiment, the one or more H₃O⁺ ion donors is/are a medium-strong acid having a pKₐ value from 0 to 2.5 at 20° C. If the pKₐ at 20° C. is 0 or less, the acid is preferably selected from sulphuric acid, hydrochloric acid, or mixtures thereof. If the pKₐ at 20° C. is from 0 to 2.5, the one or more H₃O⁺ ion donors is/are preferably selected from H₂SO₃, H₃PO₄, oxalic acid, and mixtures thereof. The one or more H₃O⁺ ion donors can also be an acid salt, for example, HSO₄⁻ or H₂PO₄⁻, being at least partially neutralized by a corresponding cation such as Li⁺, Na⁺ or K⁺, or HPO₄²⁻, being at least partially neutralised by a corresponding cation such as Li⁺, Na^{+,} K⁺, Mg²⁺ or Ca²⁺. The one or more H₃O⁺ ion donors can also be a mixture of one or more acids and one or more acid salts.

According to still another embodiment, the one or more H₃O⁺ ion donors is/are a weak acid having a pKₐ value of greater than 2.5 and less than or equal to 7, when measured at 20° C., associated with the ionisation of the first available hydrogen, and having a corresponding anion, which is capable of forming water-soluble calcium salts. Subsequently, at least one water-soluble salt, which in the case of a hydrogen-containing salt has a pKₐ of greater than 7, when measured at 20° C., associated with the ionisation of the first available hydrogen, and the salt anion of which is capable of forming water-insoluble calcium salts, is additionally provided. According to the preferred embodiment, the weak acid has a pKₐ value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the one or more H₃O⁺ ion donors is/are selected from the group consisting of hydrochloric acid, sulphuric acid, sulphurous acid, phosphoric acid, citric acid, oxalic acid, acetic acid, formic acid, and mixtures thereof. Preferably the one or more H₃O⁺ ion donors is/are selected from the group consisting of hydrochloric acid, sulphuric acid, sulphurous acid, phosphoric acid, oxalic acid, H₂PO₄⁻, being at least partially neutralised by a corresponding cation such as Li⁺, Na⁺ or K⁺, HPO₄²⁻, being at least partially neutralised by a corresponding cation such as Li⁺, Na^{+,} K⁺, Mg²⁺, or Ca²⁺ and mixtures thereof, more preferably the at least one acid is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

It is especially preferred that the surface-reacted calcium carbonate is a reaction product of natural ground calcium carbonate with carbon dioxide and phosphoric acid, wherein the carbon dioxide is formed in-situ by the phosphoric acid treatment.

The one or more H₃O⁺ ion donor, preferably phosphoric acid, can be added to the suspension as a concentrated solution or a more diluted solution. Preferably, the molar ratio of the one or more H₃O⁺ ion donor, preferably phosphoric acid, to the natural ground calcium carbonate is from 0.01 to 0.55, more preferably from 0.02 to 0.55, even more preferably 0.05 to 0.55 and most preferably 0.1 to 0.55.

It is preferred that the surface-reacted calcium carbonate is a reaction product of natural ground calcium carbonate with carbon dioxide and phosphoric acid in an aqueous medium, wherein the carbon dioxide is formed in-situ by the phosphoric acid treatment. In a more preferred embodiment, the surface-reacted calcium carbonate is a reaction product of natural ground calcium carbonate selected from the group comprising marble, chalk, limestone and mixtures thereof with carbon dioxide and phosphoric acid in an aqueous medium, wherein the carbon dioxide is formed in-situ by the phosphoric acid treatment.

In a next step, the natural ground calcium carbonate is treated with carbon dioxide.

H₃O⁺ ion donor treatment and treatment with carbon dioxide can be carried out simultaneously which is the case when a strong or medium-strong acid is used. It is also possible to carry out H₃O⁺ ion donor treatment first, e.g. with a medium strong acid having a pKₐ in the range of 0 to 2.5 at 20° C., wherein carbon dioxide is formed in situ, and thus, the carbon dioxide treatment will automatically be carried out simultaneously with the H₃O⁺ ion donor treatment, followed by the additional treatment with carbon dioxide supplied from an external source.

In a preferred embodiment, the H₃O⁺ ion donor, preferably phosphoric acid, treatment step and/or the carbon dioxide treatment step are repeated at least once, more preferably several times. According to one embodiment, the H₃O⁺ ion donor, preferably phosphoric acid, is added over a time period of at least about 5 min, preferably at least about 10 min, typically from about 10 to about 20 min, more preferably about 30 min, even more preferably about 45 min, and sometimes about 1 h or more. It is especially preferred that the H₃O⁺ ion donor, preferably phosphoric acid, is added over a time period ranging from about 10 to about 20 min, e.g. about 15 min.

Subsequent to the H₃O⁺ ion donor, preferably phosphoric acid, treatment and carbon dioxide treatment, the pH of the aqueous suspension, measured at 20° C., naturally reaches a value of greater than 6.0, preferably greater than 6.5, more preferably greater than 7.0, thereby preparing the surface-reacted natural ground calcium carbonate as an aqueous suspension having a pH of greater than 6.0, preferably greater than 6.5, more preferably greater than 7.0.

It is appreciated that the H₃O⁺ ion donor, preferably phosphoric acid, treatment and treatment with carbon dioxide can be carried over a wide temperature range. Preferably, the H₃O⁺ ion donor, preferably phosphoric acid, treatment and treatment with carbon dioxide can be carried out at room temperature or elevated temperature. For example, if the H₃O⁺ ion donor, preferably phosphoric acid, treatment and treatment with carbon dioxide is carried out at elevated temperature, the treatment is preferably in a range from 30 to 90° C., more preferably from 40 to 80° C. and most preferably from 50 to 80° C., such as from 60 to 80° C.

Further details about the preparation of the surface-reacted natural calcium carbonate are disclosed in WO0039222 A1, WO2004083316 A1, WO2005121257 A2, WO2009074492 A1, EP2264108 A1, EP2264109 A1 and US20040020410 A1, the content of these references herewith being included in the present application.

The aqueous suspension described above is dried, thereby obtaining the solid (i.e. dry or containing as little water that it is not in a fluid form) surface-reacted natural ground calcium carbonate in the form of granules or a powder.

In a preferred embodiment, the surface-reacted calcium carbonate before co-grinding has a BET specific surface area of from 1 m²/g to 200 m²/g, preferably 2 m²/g to 150 m²/g, more preferably 20 m²/g to 140 m²/g, most preferably 40 m²/g to 110 m²/g, measured using nitrogen and the BET method according to ISO 9277:2010.

It is furthermore preferred that the surface-reacted calcium carbonate particles before co-grinding have a volume median particle diameter d₅₀ (or d₅₀ (vol)) of from 0.5 to 50 µm, preferably from 0.7 to 25 µm, more preferably 0.8 to 20 µm, particularly 1 to 10 µm measured by using laser diffraction.

According to an exemplary embodiment, the surface-reacted calcium carbonate before co-grinding has
a) a volume median grain diameter d₅₀(vol) of 0.5 to 50 µm, preferably from 0.7 to 25 µm, more preferably 0.8 to 20 µm, particularly 1 to 10 µm, measured by using laser diffraction, and/or
b) a BET specific surface area of from 1 m²/g to 200 m²/g, preferably 2 m²/g to 150 m²/g, more preferably 20 m²/g to 140 m²/g, most preferably 40 m²/g to 110 m²/g, measured using nitrogen and the BET method according to ISO 9277:2010.

Preferably, the surface-reacted calcium carbonate before co-grinding has
a) a volume median grain diameter d₅₀(vol) of 0.5 to 50 µm, preferably from 0.7 to 25 µm, more preferably 0.8 to 20 µm , particularly 1 to 10 µm , measured by using laser diffraction, or
b) a BET specific surface area of from 1 m²/g to 200 m²/g, preferably 2 m²/g to 150 m²/g, more preferably 20 m²/g to 140 m²/g, most preferably 40 m²/g to 110 m²/g, measured using nitrogen and the BET method according to ISO 9277:2010.

Alternatively, the surface-reacted calcium carbonate before co-grinding has
a) a volume median grain diameter d₅₀(vol) of 0.5 to 50 µm, preferably from 0.7 to 25 µm , more preferably 0.8 to 20 µm , particularly 1 to 10 µm , measured by using laser diffraction, and
b) a BET specific surface area of from 1 m²/g to 200 m²/g, preferably 2 m²/g to 150 m²/g, more preferably 20 m²/g to 140 m²/g, most preferably 40 m²/g to 110 m²/g, measured using nitrogen and the BET method according to ISO 9277:2010.

It may furthermore be preferred that the surface-reacted calcium carbonate particles before co-grinding have a volume particle diameter d₉₈ (or d₉₈ (vol)) of from 2 to 150 µm, preferably from 3 to 100 µm , more preferably 6 to 80 µm , even more preferably from 8 to 60 µm, and most preferably from 10 to 30 µm.

Thus, the surface-reacted calcium carbonate before co-grinding preferably has
a) a volume median grain diameter d₅₀(vol) of 0.5 to 50 µm, preferably from 0.7 to 25 µm, more preferably 0.8 to 20 µm , particularly 1 to 10 µm , measured by using laser diffraction, and
b) a BET specific surface area of from 1 m²/g to 200 m²/g, preferably 2 m²/g to 150 m²/g, more preferably 20 m²/g to 140 m²/g, most preferably 40 m²/g to 110 m²/g, measured using nitrogen and the BET method according to ISO 9277:2010, and
c) a volume particle diameter d₉₈(vol) of from 2 to 150 µm , preferably from 3 to 100 µm, more preferably 6 to 80 µm, even more preferably from 8 to 60 µm, and most preferably from 10 to 30 µm.

The value dₓ represents the diameter relative to which x % of the particles have diameters less than dₓ. This means that the d₉₈ value is the particle size at which 98 % of all particles are smaller. The d₉₈ value is also designated as “top cut”. The dₓ values may be given in volume or weight percent. The d₅₀(wt) value is thus the weight median particle size, i.e. 50 wt.-% of all grains are smaller than this size, and the d₅₀ (vol) value is the volume median particle size, i.e. 50 vol.% of all grains are smaller than this particle size.

The “particle size” of surface-reacted calcium carbonate herein is described as volume-based particle size distribution. Furthermore, the “particle size” of surface-reacted calcium carbonate in the meaning of the present invention refers to the primary particle size.

Volume median particle diameter d₅₀ was evaluated using a Malvern Mastersizer 2000 or 3000 Laser Diffraction System. The d₁₀, d₅₀ or d₉₈ value, measured using a Malvern Mastersizer 2000 or 3000 Laser Diffraction System, indicates a diameter value such that 10%, 50 % or 98 % by volume, respectively, of the particles have a diameter of less than this value. The raw data obtained by the measurement are analysed using the Mie theory, with a particle refractive index of 1.57 and an absorption index of 0.005.

Throughout the present invention, the volume-based particle size distribution is determined by laser diffraction.

Preferably, the surface-reacted calcium carbonate has an intra-particle intruded specific pore volume within the range from 0.15 to 1.60 cm³/g, preferably from 0.30 to 1.50 cm³/g, more preferably from 0.30 to 1.40 cm³/g, and most preferably from 0.30 to 1.35 cm³/g calculated from a mercury intrusion porosimetry measurement.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 µm (~ nm). The equilibration time used at each pressure step is 20 seconds. The sample material is sealed in a 5 cm³ chamber powder penetrometer for analysis. The data are corrected for mercury compression, penetrometer expansion and sample material compression using the software Pore-Comp (Gane, P.A.C., Kettle, J.P., Matthews, G.P. and Ridgway, C.J., “Void Space Structure of Compressible Polymer Spheres and Consolidated Calcium Carbonate Paper-Coating Formulations”, Industrial and Engineering Chemistry Research, 35(5), 1996, p1753-1764.).

The total pore volume seen in the cumulative intrusion data can be separated into two regions with the intrusion data from 214 µm down to about 1 - 4 µm showing the coarse packing of the sample between any agglomerate structures contributing strongly. Below these diameters lies the fine interparticle packing of the particles themselves. If they also have intraparticle pores, then this region appears bi modal, and by taking the specific pore volume intruded by mercury into pores finer than the modal turning point, i.e. finer than the bi-modal point of inflection, we thus define the specific intraparticle pore volume. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

The intra-particle pore size of the surface-reacted calcium carbonate preferably is in a range of from 0.004 to 1.6 µm, more preferably in a range of between 0.005 to 1.3 µm, especially preferably from 0.006 to 1.15 µm and most preferably of 0.007 to 1.0 µm, determined by mercury porosimetry measurement.

The surface-reacted calcium carbonate of the present invention comprises a water-insoluble, at least partially crystalline hydroxyapatite (Ca₅(PO₄)₃OH), which is formed at the surface of the natural ground calcium carbonate. According to one embodiment, the water-insoluble, at least partially crystalline calcium phosphate salt covers the surface of the natural ground calcium carbonate at least partially, preferably completely. According to one embodiment, the surface-reacted calcium carbonate provides a ratio of hydroxyapatite to calcium carbonate in the range of from 1:99 to 99:1 by weight. Preferably, the surface-reacted calcium carbonate provides a ratio of hydroxyapatite to calcium carbonate in the range of from 20:80 to 99:1 by weight, more preferably 40:60 to 95:5 by weight, and most preferably in the range from 45:55 to 90:10 by weight. The ratio of hydroxyapatite to calcium carbonate is determined by XRD.

As stated above, the surface-reacted calcium carbonate is provided in dry form for co-grinding with the one or more solid active(s).

In one embodiment, the carrier material of the co-ground active(s) comprising product is preferably free of materials differing from surface-reacted calcium carbonate. In particular, the carrier material is free of silicates such as silica gel, calcium silicate, magnesium silicate, magnesium trisilicate or magnesium aluminometasilicate, clays such as kaolin, talc, titanium dioxide, zinc oxide, calcium hydrogen phosphate, zeolites lactose, lactose derivatives, starch, starch derivatives, treated starch, chitin; cellulose and derivatives thereof, e.g. microcrystalline cellulose (e.g. Avicel), cyclodextrins, phospholipids, magnesium carbonate, magnesium phosphate, magnesium oxide, maltodextrin, calcium sulphate, dextrates, dextrin, dextrose, hydrogenated vegetable oil, sodium chloride, potassium chloride and mixtures thereof.

Thus, the carrier material preferably consists of the surface-reacted calcium carbonate.

It is a further requirement that the co-ground active(s) comprising product comprises one or more at least partially X-ray amorphous active(s) selected from the group comprising, preferably consisting of, pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding.

Preferably, the co-ground active(s) comprising product comprises one or more at least partially X-ray amorphous active(s) selected from the group comprising, preferably consisting of, pharmaceutical active(s) or inactive precursor thereof, nutraceutical active(s) or inactive precursor thereof, veterinary active(s) and/or inactive precursor thereof, agricultural active(s) or inactive precursor thereof, and mixtures thereof, wherein the one or more active(s) is/are rendered at least partially X-ray amorphous during co-grinding.

In one embodiment, the co-ground active(s) comprising product comprises one or more at least partially X-ray amorphous active(s) selected from the group comprising, preferably consisting of, pharmaceutical active(s) or inactive precursor thereof, nutraceutical active(s) or inactive precursor thereof, veterinary active(s) and/or inactive precursor thereof, agricultural active(s) or inactive precursor thereof, and mixtures thereof, wherein the one or more active(s) is/are rendered at least partially X-ray amorphous during co-grinding.

For example, the co-ground active(s) comprising product comprises one or more at least partially X-ray amorphous active(s) selected from the group comprising, preferably consisting of, pharmaceutical active(s) or inactive precursor thereof, nutraceutical active(s) or inactive precursor thereof, veterinary active(s) and/or inactive precursor thereof, agricultural active(s) or inactive precursor thereof, and mixtures thereof, wherein the one or more active(s) is/are rendered at least partially X-ray amorphous during co-grinding.

In one embodiment, the co-ground active(s) comprising product comprises one or more at least partially X-ray amorphous active(s) selected from pharmaceutical active(s) or inactive precursor thereof, preferably pharmaceutical active(s), wherein the one or more active(s) is/are rendered at least partially X-ray amorphous during co-grinding. Alternatively, the co-ground active(s) comprising product comprises one or more at least partially X-ray amorphous active(s) selected from nutraceutical active(s) or inactive precursor thereof, preferably nutraceutical active(s), wherein the one or more active(s) is/are rendered at least partially X-ray amorphous during co-grinding. Alternatively, the co-ground active(s) comprising product comprises one or more at least partially X-ray amorphous active(s) selected from veterinary active(s) or inactive precursor thereof, preferably veterinary active(s), wherein the one or more active(s) is/are rendered at least partially X-ray amorphous during co-grinding. Alternatively, the co-ground active(s) comprising product comprises one or more at least partially X-ray amorphous active(s) selected from agricultural active(s) or inactive precursor thereof, preferably agricultural active(s), wherein the one or more active(s) is/are rendered at least partially X-ray amorphous during co-grinding.

It is preferred that the co-ground active(s) comprising product comprises one or more at least partially X-ray amorphous active(s) selected from pharmaceutical active(s) or inactive precursor thereof, preferably pharmaceutical active(s), wherein the one or more active(s) is/are rendered at least partially X-ray amorphous during co-grinding.

In one embodiment of the present invention, the co-ground active(s) comprising product comprises one at least partially X-ray amorphous active. Alternatively, the co-ground active(s) comprising product comprises two or more at least partially X-ray amorphous active(s). For example, the co-ground active(s) comprising product comprises two or three at least partially X-ray amorphous active(s).

Preferably, the co-ground active(s) comprising product comprises one at least partially X-ray amorphous active.

It is to be noted that the one or more active(s) which may be used in the present invention generally are those well-known in the kind of products to be prepared.

The term “active” or “active ingredient” in the meaning of the present invention refers to a substance having a specific effect in an organism and causing a specific reaction in humans, animals, microorganisms and/or plants.

It is preferred that the at least one active and/or inactive precursor thereof is/are provided in solid form before co-grinding.

The term “solid” in the meaning of the present invention refers to a particulate compound, i.e. a non-gaseous and non-liquid compound, comprising or consisting of the one or more active(s) which is/are solid at room temperature, i.e. about 21° C.

Thus, it is preferred that the one or more solid active(s), i.e. before co-grinding, has/have a melting point Tm of at least 50° C., more preferably at least 60° C. and most preferably in the range from 60 to 400° C.

It is appreciated that the one or more at least partially X-ray amorphous active(s) is/are selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof.

The active may be also provided in form of the inactive precursor thereof, which will be activated at a later stage. The activation of such inactive precursors is known to the skilled person and commonly in use, e.g. activation in the stomach and/or gastro-intestinal pathway-such as acidic activation, tryptic, chimotryptic or pepsinogenic cleavage. It lies within the understanding of the skilled person that the mentioned activation methods are of mere illustrative character and are not intended to be of limiting character.

According to one embodiment, the one or more active(s) is/are selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, may be of synthetic origin, semi-synthetic origin, natural origin and combinations thereof.

For example, the one or more at least partially X-ray amorphous active(s) may be a chiral compound. Thus, the one or more at least partially X-ray amorphous active(s) encompass the (R)-enantiomer, (S)-enantiomer and mixtures thereof, e.g. the racemic mixture.

Additionally or alternatively, the one or more at least partially X-ray amorphous active(s) may be an isomeric compound. Thus, the one or more at least partially X-ray amorphous active(s) encompass the (Z)-isomer, (E)-isomer and mixtures thereof.

Nutraceutical active(s) and/or inactive precursor thereof preferably include any compound that provides prophylactic and/or therapeutic properties when administered to humans and/or animals. It is appreciated that nutraceutical actives may have the same effects and may encompass the same compounds as pharmaceutical actives. However, dietary supplements and food additives are typically considered as nutraceutical actives. Examples of nutraceutical actives include, but are not limited to, vitamins, minerals, phytochemicals, probiotics, prebiotics, sugars and other substances such as curcumine, resveratrol and isoflavones.

For example, vitamin A, vitamin B1, vitamin B6, vitamin B12, vitamin B2, vitamin B6, vitamin D, vitamin K, thiamine, riboflavin, biotin, folic acid, niacin, alpha lipoic acid, dihydrolipoic acid, curcumin, xanthophylls, beta cryptoxanthin, lycopene, lutein, zeaxanthin, astaxanthin, beta-carotene, carotenes, mixed carotenoids, polyphenols, flavonoids, sodium salts, potassium salts, calcium salts, magnesium salts, sulphur, choline, and/or phytochemicals such as carotenoids, chlorophyll, chlorophyllin, flavanoids, anthocyanins, cyaniding, delphinidin, malvidin, pelargonidin, peonidin, petunidin, flavanols, catechin, epicatechin, epigallocatechin, epigallocatechingallate, theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, soy isoflavones, and combinations thereof, may be used. Examples of nutraceutical active(s) and/or inactive precursor thereof that can be used as active s) are set forth in U.S. Pat. Application Publication Nos. 20030157213 A1, 20030206993 and 20030099741 A1 which are incorporated in their entirety herein by reference for all purposes.

Nutraceutical active(s) and/or inactive precursor thereof may also include (trace) minerals such as salts (preferably organic salts) of manganese, zinc, copper, fluorine, molybdenum, iodine, iron, cobalt, chromium, selenium, phosphorous, magnesium, potassium, sodium, and combinations thereof or enzymes such as coenzyme Q10, biotin, pepsin, phytase, trypsin, lipases, proteases, cellulases, lactase and combinations thereof.

Nutraceutical active(s) and/or inactive precursor thereof may also include sugars such as sucrose, glucose, fructose, palm sugar, coconut blossom sugar, sugar alcohols and combinations thereof or artificial sweeteners such as aspartame, acesulfame potassium, advantame, aspartame-acesulfame salt, cyclamate, neotame, neohesperidin, sacchari, sucralose and combinations thereof.

Agricultural active(s) and/or inactive precursor thereof are preferably any known herbicide, insecticide, insect growth regulator, nematicide, termiticide, molluscicide, piscicide, avicide, rodenticide, predacide, bactericide, insect repellent, animal repellent, antimicrobial, fungicide, disinfectant (antimicrobial), and sanitizer known to the skilled person. For example, dimethomorph can be used as agricultural active.

It is to be noted that the pharmaceutical active(s) and/or inactive precursor thereof, may be any such compound known to the skilled person.

Pharmaceutical active(s) thus include any compound that provides prophylactic and/or therapeutic properties when administered to humans and/or animals. Examples include, but are not limited to, pharmaceutical actives, therapeutic actives, veterinarian actives, and growth regulators.

The pharmaceutical active(s) and/or inactive precursor thereof is preferably selected from the group comprising pharmaceutical active(s) or inactive precursor thereof of synthetic origin, semi-synthetic origin, natural origin and combinations thereof.

Thus, a pharmaceutical active refers to pharmaceutical actives which are of synthetic origin, semi-synthetic origin, natural origin and combinations thereof. Further, a pharmaceutical inactive precursor of the pharmaceutical active refers to pharmaceutical inactive precursors which are of synthetic origin, semi-synthetic origin, natural origin and combinations thereof and will be activated at a later stage to the respective pharmaceutical active.

The pharmaceutical active(s) and/or inactive precursor thereof can be an anti-inflammatory agent. Such agents may include, but are not limited to, non- steroidal anti- inflammatory agents or NSAIDs, such as propionic acid derivatives or salts; acetic acid derivatives or salts; fenamic acid derivatives and salts; biphenylcarboxylic acid derivatives and salts; and oxicams. All of these NSAIDs are fully described in U.S. Pat. Number 4,985,459 to Sunshine et al., incorporated by reference herein in its entirety as to the description of such NSAIDs. Examples of useful NSAIDs include acetylsalicylic acid, ibuprofen, naproxen, benoxaprofen, flurbiprofen, fenoprofen, fenbufen, ketoprofen, indoprofen, pirprofen, carprofen, oxaprozin, pranoprofen, microprofen, tioxaprofen, suprofen, alminoprofen, tiaprofenic acid, fluprofen, bucloxic acid, the salts thereof and mixtures thereof. Also useful are the steroidal anti-inflammatory drugs such as hydrocortisone and the like, and COX-2 inhibitors such as meloxicam, celecoxib, rofecoxib, valdecoxib, etoricoxib or mixtures thereof. Mixtures of any of the above anti-inflammatories may be used.

Other materials that can be used as pharmaceutical active(s) and/or inactive precursor thereof include commonly known mouth and throat products. These products include, but are not limited to, upper respiratory agents such as phenylephrine, diphenhydramine, dextromethorphan, bromhexine and chlorpheniramine, gastrointestinal agents such as famotidine, loperamide and simethicone, antifungals such as miconazole nitrate, antibiotics and analgesics such as ketoprofen and fluribuprofen.

The pharmaceutical active(s) and/or inactive precursor thereof may be also selected from sodium pyrosulphite, butylhydroxytoluene, butylated hydroxyanisole.

The pharmaceutical active(s) and/or inactive precursor thereof may be also selected from ephedrine, magaldrate, pseudoephedrine, sildenafil, xylocaine, benzalconium chloride, caffeine, phenylephrine, amfepramone, orlistat, sibutramine, acetaminophen, aspirin, glitazones, metformin, chlorpromazine, dimenhydrinat, domperidone, meclozine, metoclopramide, odansetron, prednisolone, promethazine, acrivastine, cetirizine, cinnarizine, clemastine, cyclizine, desloratadine, dexchlorpheniramine, dimenhydrinate, ebastine, fexofenadine, ibuprofen, levolevoproricin, loratadine, meclozine, mizolastine, promethazine, miconazole, chlorhexidine diacetate, decapeptide KSL, aluminium fluoride, aminochelated calcium, ammonium fluoride, ammonium fluorosilicate, ammonium monofluorphosphate, calcium gluconate, calcium glycerophosphate, calcium lactate, calcium monofluorphosphate, carbamide, cetyl pyridinium chloride, chlorhexidine, chlorhexidine digluconate, chlorhexidine chloride, chlorhexidine diacetate, CPP caseine phospho peptide, hexetedine, octadecentyl ammonium fluoride, potassium fluorosilicate, potassium monofluorphosphate, sodium fluorosilicate, sodium monofluorphosphate, sodium tri polyphosphate, stearyl trihydroxyethyl propylenediamine dihydrofluoride, strontium chloride, tetra potassium pyrophosphate, tetra sodium pyrophosphate, tripotassium orthophosphate, trisodium orthophosphate, alginic acid, sildenafil, tadalafil, vardenafil, yohimbine, cimetidine, nizatidine, ranitidine, acetylsalicylic acid, clopidogrel, acetylcysteine, bromhexine, codeine, dextromethorphan, diphenhydramine, noscapine, phenylpropanolamine, vitamin D, simvastatin, bisacodyl, lactitol, lactulose, sodium picosulphate, senna glycosides, benzocaine, lidocaine, tetracaine, almotriptan, eletriptan, naratriptan, rizatriptan, sumatriptan, zolmitriptan, calcium salts of organic acids, chromium salts of organic acids, copper salts of organic acids, iodine salts of organic acids, magnesium salts of organic acids, manganese salts of organic acids, molybdenium salts of organic acids, phosphor salts of organic acids, selenium salts of organic acids, zinc salts of organic acids, chloramine, metronidazole, triamcinolonacetonide, benzethonium chl., cetyl pyrid. chl., chlorhexidine, fluoride salts, lidocaine, amphotericin, miconazole, nystatin, ginkgo biloba, ginseng, ginger, purple coneflower, saw palmetto, cetirizine, levocetirizine, loratadine, diclofenac, flurbiprofen, acrivastine pseudoephedrine, loratadine pseudoephedrine, glucosamine, hyaluronic acid, decapeptide KSL-W, decapeptide KSL, resveratrol, misoprostol, bupropion, ondansetron HCl, esomeprazole, lansoprazole, omeprazole, pantoprazole, rabeprazole, bacteria and the like, loperamide, simethicone, acetylsalicylic acid and others, sucralfate, clotrimazole, fluconazole, itraconazole, ketoconazole, terbinafine, allopurinol, probenecid, atorvastatin, fluvastatin, lovastatin, nicotinic acid, pravastatin, rosuvastatin, simvastatin, pilocarpine, naproxen, alendronate, etidronate, raloxifene, risedronate, benzodiazepines, disulphiram, naltrexone, buprenorphine, codeine, dextropropoxyphene, fentanyl, hydromorphone, ketobemidone, ketoprofen, methadone, morphine, naproxen, nicomorphine, oxycodone, pethidine, tramadol, amoxicillin, ampicillin, azithromycin, ciprofloxacin, clarithromycin, doxycyclin, erythromycin, fusidic acid, lymecycline, metronidazole, moxifloxacin, ofloxacin, oxytetracycline, phenoxymethylpenicillin, rifamycins, roxithromycin, sulphamethizole, tetracycline, trimethoprim, vancomycin, acarbose, glibenclamide, gliclazide, glimepiride, glipizide, insulin, repaglinide, tolbutamide, oseltamivir, aciclovir, famciclovir, penciclovir, valganciclovir, amlopidine, diltiazem, felodipine, nifedipine, verapamil, finasteride, minoxidil, cocaine, buphrenorphin, clonidine, methadone, naltrexone, calcium antagonists, clonidine, ergotamine, β-blockers, aceclofenac, celecoxib, dexiprofen, etodolac, indometacin, ketoprofen, ketorolac, lornoxicam, meloxicam, nabumetone, oiroxicam, parecoxib, phenylbutazone, piroxicam, tiaprofenic acid, tolfenamic acid, aripiprazole, chlorpromazine, chlorprothixene, clozapine, flupentixol, fluphenazine, haloperidol, lithium citrate, melperone, penfluridol, periciazine, perphenazine, pimozide, pipamperone, prochlorperazine, risperidone, thioridizin, fluconazole, itraconazole, ketoconazole, voriconazole, opium, benzodiazepines, hydroxycine, meprobamate, phenothiazine, aluminiumaminoacetate, esomeprazole, famotidine, magnesium oxide, nizatide, omeprazole, pantoprazole, fluconazole, itraconazole, ketoconazole, metronidazole, amphetamine, atenolol, bisoprolol fumarate, metoprolol, pindolol, propranolol, auranofin, and bendazac.

Further examples of useful pharmaceutical active(s) and/or inactive precursor thereof can include active ingredients selected from the therapeutical groups comprising: Analgesic, Anaesthetic, Antipyretic, Anti-allergic, Anti-arrhythmic, Appetite suppressant, Antifungal, Anti-inflammatory, Broncho dilator, Cardiovascular drugs, Coronary dilator, Cerebral dilator, Peripheral vasodilator, Anti-infective, Psychotropic, Anti-manic, Stimulant, Antihistamine, Laxative, Decongestant, Gastro-intestinal sedative, Sexual dysfunction agent, Disinfectants, Anti-diarrhoeal, Anti-anginal substance, Vasodilator, Anti-hypertensive agent, Vasoconstrictor, Migraine treating agent, Antibiotic, Tranquilizer, Antipsychotic, Anti-tumour drug, Anticoagulant, Antithrombotic agent, Hypnotic, Sedative, Anti-emetic, Anti-nauseant, Anticonvulsant, Neuromuscular agent, Hyper and hypoglycaemic, Thyroid and antithyroid, Diuretic, Antispasmodic, Uterine relaxant, Anti-obesity agent, Anorectic, Spasnolytics, Anabolic agent, Erythropoietic agent, Anti-asthmatic, Expectorant, Cough suppressant, Mucolytic, Anti-uricemic agent, Dental vehicle, Breath freshener, Antacid, Anti-diuretic, Anti-flatulent, Betablocker, Teeth Whitener, Enzyme, Co-enzyme, Protein, Energy booster, Probiotics, Prebiotics, NSAID, Anti-tussives, Decongestants, Anti-histamines, Expectorants, Anti-diarrhoeals, Hydrogen antagonists, Proton pump inhibitors, General nonselective CNS depressants, General nonselective CNS stimulants, Selectively CNS function modifying drugs, Antiparkinsonism, Narcotic-analgetics, Analgetic-antipyretics, Psychopharmacological drugs, and Sexual dysfunction agents.

Examples of useful pharmaceutical active(s) and/or inactive precursor thereof may also include: Casein glyco-macro-peptide (CGMP), Triclosan, Cetyl pyridinium chloride, Domiphen bromide, Quaternary ammonium salts, zinc components, Sanguinarine, Alexidine, Octonidine, EDTA, Aspirin, Acetaminophen, Ibuprofen, Ketoprofen, Diflunisal, Fenoprofen calcium, Naproxen, Tolmetin sodium, Indomethacin, Benzonatate, Caramiphen edisylate, Menthol, Dextromethorphan hydrobromide, Theobromine hydrochloride, Chlophendianol Hydrochloride, Pseudoephedrine Hydrochloride, Phenylephrine, Phenylpropanolamine, Pseudoephedrine sulphate, Brompheniramine maleate, Chlorpheniramine- maleate, Carbinoxamine maleate, Clemastine fumarate, Dexchlorpheniramine maleate, Dephenhydramine hydrochloride, Diphenpyralide hydrochloride, Azatadine maleate, Diphenhydramine citrate, Doxylamine succinate, Promethazine hydrochloride, Pyrilamine maleate, Tripellenamine citrate, Triprolidine hydrochloride, Acrivastine, Loratadine, Brompheniramine, Dexbrompheniamine, Guaifenesin, Terpin hydrate, Loperamide, Famotidine, Ranitidine, Omeprazole, Lansoprazole, solid (long chain) aliphatic alcohols, Barbiturates, caffeine, strychnine, Picrotoxin, Pentyenetetrazol, Phenyhydantoin, Phenobarbital, Primidone, Carbamazapine, Etoxsuximide, Methsuximide, Phensuximide, Trimethadione, Diazepam, Benzodiazepines, Phenacemide, Pheneturide, Acetazolamide, Sulthiame, bromide, Levodopa, Amantadine, Morphine, Heroin, Hydromorphone, Metopon, Oxymorphone, Levophanol, Codeine, Hydrocodone, Xycodone, Nalorphine, Naloxone, Naltrexone, Salicylates, Phenylbutazone, Indomethacin, Phenacetin, Chlorpromazine, Methotrimeprazine, Haloperidol, Clozapine, Reserpine, Imipramine, Tranylcypromine, Phenelzine, Lithium, Sildenafil citrate, Tadalafil, and Vardenafil CL.

Examples of useful pharmaceutical active(s) and/or inactive precursor thereof may include actives selected from the groups of ace-inhibitors, antianginal drugs, anti- arrhythmias, anti-asthmatics, anti-cholesterolemics, analgesics, anaesthetics, anticonvulsants, anti-depressants, antidiabetic agents, anti-diarrhoea preparations, antidotes, anti-histamines, anti-hypertensive drugs, anti-inflammatory agents, anti-lipid agents, anti- manics, anti-nauseants, anti-stroke agents, anti-thyroid preparations, anti-tumour drugs, anti- viral agents, acne drugs, alkaloids, amino acid preparations, anti-tussives, anti- uricemic drugs, anti-viral drugs, anabolic preparations, systemic and non-systemic anti- infective agents, anti-neoplasties, antiparkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, which is currently marketed as Viagra™, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocriptine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumour drugs, anti-coagulants, antithrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of useful pharmaceutical active(s) and/or inactive precursor thereof contemplated can also include antacids, H2-antagonists, and analgesics. For example, antacid dosages can be prepared using the ingredients calcium carbonate alone or in combination with magnesium hydroxide, and/or aluminium hydroxide. Moreover, antacids can be used in combination with H2-antagonists.

Analgesics include opiates and opiate derivatives, such as Oxycontin™, ibuprofen, aspirin, acetaminophen, and combinations thereof that may optionally include caffeine.

Other useful pharmaceutical active(s) and/or inactive precursor thereof can include anti-diarrhoeals such as Immodium™ AD, anti-histamines, anti-tussives, decongestants, vitamins, and breath fresheners. Also contemplated for use herein are anxiolytics such as Xanax™; anti-psychotics such as Clozaril™ and Haldol™; non-steroidal anti-inflammatories (NSAID’s) such as ibuprofen, naproxen sodium, Voltaren™ and Lodine™, anti-histamines such as Claritin™, Hismanal™, Relafen™, and Tavist™; antiemetics such as Kytril™ and Cesamet™; bronchodilators such as Bentolin™, Proventil™; anti-depressants such as Prozac™, Zoloft™, and Paxil™; anti-migraines such as Imigra™, ACE-inhibitors such as Vasotec™, Capoten™ and Zestril™; anti- Alzheimer’s agents, such as Nicergoline™; and CaH-antagonists such as Procardia™, Adalat™, and Calan™.

The popular H2-antagonists which are contemplated for use in the present invention include cimetidine, ranitidine hydrochloride, famotidine, nizatidine, ebrotidine, mifentidine, roxatidine, pisatidine and aceroxatidine.

Active antacid ingredients can include, but are not limited to, the following: aluminium hydroxide, dihydroxyaluminium aminoacetate, aminoacetic acid, aluminium phosphate, dihydroxyaluminium sodium carbonate, bicarbonate, bismuth aluminate, bismuth carbonate, bismuth subcarbonate, bismuth subgallate, bismuth subnitrate, bismuth subsilysilate, calcium phosphate, citrate ion (acid or salt), amino acetic acid, hydrate magnesium aluminate sulphate, magaldrate, magnesium aluminosilicate, magnesium carbonate, magnesium glycinate, magnesium hydroxide, magnesium oxide, magnesium trisilicate, milk solids, aluminium mono-or dibasic calcium phosphate, tricalcium phosphate, potassium bicarbonate, sodium tartrate, sodium bicarbonate, magnesium aluminosilicates, tartaric acids and salts.

In some embodiments, the pharmaceutical active(s) and/or inactive precursor thereof can be selected from analgesics/anaesthetics such as menthol, phenol, hexylresorcinol, benzocaine, dyclonine hydrochloride, salicyl alcohol, and combinations thereof. In some embodiments, the pharmaceutical active(s) and/or inactive precursor thereof can be selected from demulcents such as slippery elm bark, pectin, gelatin, and combinations thereof. In some embodiments, the pharmaceutical active(s) and/or inactive precursor thereof can be selected from antiseptic ingredients such as cetylpyridinium chloride, domiphen bromide, dequalinium chloride and combinations thereof.

In some embodiments, the pharmaceutical active(s) and/or inactive precursor thereof can be selected from antitussive ingredients such as chlophedianol hydrochloride, codeine, codeine phosphate, codeine sulphate, dextromethorphan, dextromethorphan hydrobromide, diphenhydramine citrate, and diphenhydramine hydrochloride, and combinations thereof.

In some embodiments, the pharmaceutical active(s) and/or inactive precursor thereof can be selected from throat soothing agents such as propolis, menthol and combinations thereof. In still other embodiments, the pharmaceutical active(s) and/or inactive precursor thereof can be selected from cough suppressants. Such cough suppressants can fall into two groups: those that alter the texture or production of phlegm such as mucolytics and expectorants; and those that suppress the coughing reflex such as codeine (narcotic cough suppressants), antihistamines, dextromethorphan and isoproterenol (non-narcotic cough suppressants).

In still other embodiments, the pharmaceutical active(s) and/or inactive precursor thereof can be an antitussive selected from the group comprising codeine, dextromethorphan, dextrorphan, diphenhydramine, hydrocodone, noscapine, oxycodone, pentoxyverine and combinations thereof. In some embodiments, the pharmaceutical active(s) and/or inactive precursor thereof can be selected from antihistamines such as acrivastine, azatadine, brompheniramine, chlorpheniramine, clemastine, cyproheptadine, dexbrompheniramine, dimenhydrinate, diphenhydramine, doxylamine, hydroxyzine, meclizine, phenindamine, phenyltoloxamine, promethazine, pyrilamine, tripelennamine, triprolidine and combinations thereof. In some embodiments, the pharmaceutical active(s) and/or inactive precursor thereof can be selected from non-sedating antihistamines such as astemizole, cetirizine, ebastine, fexofenadine, loratidine, terfenadine, and combinations thereof.

As regards the veterinary active(s) and/or inactive precursor thereof, it is to be noted that each pharmaceutical active(s) and/or inactive precursor thereof may be used as the veterinary active(s) and/or inactive precursor thereof as long as they are intended for the treatment of animals.

It is to be noted that the pharmaceutical active(s) and/or inactive precursor thereof or veterinary active(s) and/or inactive precursor thereof may be also in form of a corresponding salt, such as a sodium or potassium salt.

The actives described above are only included in the present invention as far as they are solid at room temperature, i.e. about 21° C., before co-grinding.

It is appreciated that the one or more active(s) is/are rendered at least partially X-ray amorphous during co-grinding. Preferably, the one or more active(s) is/are rendered substantially X-ray amorphous during co-grinding. That is to say, after co-grinding the one or more active(s) has/have a reduced crystallinity compared to the same active(s) before co-grinding with the surface-reacted calcium carbonate.

For example, after co-grinding the crystallinity of the one or more active(s) is reduced crystallinity compared to the same active(s) before co-grinding by more than 6 wt.-%, preferably more than 70 wt.-%, and most preferably more than 80 wt.-%, based on the total weight of the one or more active(s).

Thus, it is preferred that the one or more at least partially X-ray amorphous active(s) in the co-ground active(s) comprising product has/have a crystallinity of less than 50 wt.-%, preferably of less than 40 wt.-%, more preferably of less than 30 wt.-% and most preferably of less than 20 wt.-%, based on the total weight of the one or more at least partially X-ray amorphous active.

Additionally or alternatively, the one or more at least partially X-ray amorphous active(s) has/have an amorphous fraction of more than 50 wt.-%, preferably of more than 60 wt.-%, more preferably of more than 70 wt.-% and most preferably of more than 80 wt.-%, based on the total weight of the one or more at least partially X-ray amorphous active.

In one embodiment, the one or more at least partially X-ray amorphous active(s) in the co-ground active(s) comprising product has/have a crystallinity of less than 40 wt.-%, preferably of less than 30 wt.-% and most preferably of less than 20 wt.-%, based on the total weight of the one or more at least partially X-ray amorphous active.

Additionally or alternatively, the one or more at least partially X-ray amorphous active(s) has/have an amorphous fraction of more than 60 wt.-%, more preferably of more than 70 wt.-% and most preferably of more than 80 wt.-%, based on the total weight of the one or more at least partially X-ray amorphous active.

It is to be noted that a crystallinity as described above for the at least partially X-ray amorphous active(s) in the co-ground active(s) comprising product cannot be obtained by a process comprising the steps of loading surface-reacted calcium carbonate with at least one active ingredient and/or inactive precursor thereof, compacting the loaded surface-reacted calcium carbonate obtained by means of a roller compacter at a compaction pressure in the range from 1 to 30 kN/cm into a compacted form; and milling the compacted form into granules as for example described in European patent application EP3260114 A1.Furthermore, the one or more at least partially X-ray amorphous active(s) in the co-ground active(s) comprising product, i.e. after co-grinding, has/have preferably a melting point Tm which is reduced compared to the one or more solid active(s), i.e. before co-grinding. In one embodiment, the one or more at least partially X-ray amorphous active(s) in the co-ground active(s) comprising product, i.e. after co-grinding, has/have a melting point Tm of at least 30° C., more preferably at least 35° C., even more preferably at least 40° C., and most preferably at least 45° C. or 50° C. For example, the one or more at least partially X-ray amorphous active(s) in the co-ground active(s) comprising product, i.e. after co-grinding, has/have a melting point Tm in the range from 30 to 350° C., more preferably from 35 to 350° C., even more preferably from 40 to 350° C., and most preferably from 45 to 300° C. or from 50 to 300° C.

As already mentioned above, the co-ground active(s) comprising product may comprise additive(s). The term “additive(s)” may comprise one or more additive(s). For example, the co-ground active(s) comprising product comprises one additive. Alternatively, the co-ground active(s) comprising product comprises two or more additives, preferably two additives. In one embodiment, the co-ground active(s) comprising product comprises one additive.

It is, however, preferred that the co-ground active(s) comprising product is free of additive(s) and thus consists of the carrier material being surface-reacted calcium carbonate, and the one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding.

If present, the additive(s) may be selected from the comprising, preferably consisting of, antioxidants, disintegrants, binders, diluents, lubricants, film forming agents, adhesives, buffers, adsorbents, natural or synthetic scenting agents, natural or synthetic flavouring agents, natural or synthetic colouring agents, natural or synthetic sweeteners, natural or synthetic odour-masking agents, natural or synthetic flavouring or taste-masking agents, and/or mixtures thereof. For example, the additive may be an outer-phase lubricant. Said outer-phase lubricant can be selected from the group comprising lecithin, polyoxyethylene stearate, polyoxyethylene sorbitan fatty acid esters, fatty acid salts, mono and diacetyl tartaric acid esters of mono and diglycerides of edible fatty acids, citric acid esters of mono and diglycerides of edible fatty acids, saccharose esters of fatty acids, polyglycerol esters of fatty acids, polyglycerol esters of interesterified castor oil acid (E476), sodium stearoyllactylate, magnesium and/or calcium stearate, hydrogenated vegetable oils, stearic acid, sodium lauryl sulphate, magnesium lauryl sulphate, colloidal silica, talc and combinations thereof. Preferably, said outer-phase lubricant is magnesium and/or calcium stearate, more preferably magnesium stearate.

The co-ground active(s) comprising product is prepared by co-grinding the carrier material being surface-reacted calcium carbonate, and one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof.

Thus, the co-ground active(s) comprising product is preferably prepared by a method comprising, preferably consisting of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,

wherein the co-grinding in step c) is carried out in the absence of solvent(s).

If additive(s) are present, the co-ground active(s) comprising product is preferably prepared by a method comprising, preferably consisting of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,
d) contacting the surface-reacted calcium carbonate of step a) before, during or after co-grinding step c) with additive(s),

wherein the co-grinding in step c) and the contacting in step d) are carried out in the absence of solvent(s).

In order to improve the homogeneity of the co-ground active(s) comprising product, the surface-reacted calcium carbonate and the one or more solid active(s) may be mixed before co-grinding step c) is carried out. Thus, a pre-mixed mixture of the surface-reacted calcium carbonate and the one or more solid active(s) is subjected to co-grinding step c).

If such mixing step is present, the co-ground active(s) comprising product is preferably prepared by a method comprising, preferably consisting of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,

wherein the co-grinding in step c) is carried out in the absence of solvent(s), and

wherein the surface-reacted calcium carbonate of step a) and the one or more solid active(s) of step b) are mixed before co-grinding step c) is carried out.

Furthermore, if additive(s) are present, the co-ground active(s) comprising product is preferably prepared by a method comprising, preferably consisting of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,
d) contacting the surface-reacted calcium carbonate of step a) before, during or after co-grinding step c) with additive(s),

wherein the co-grinding in step c) and the contacting in step d) are carried out in the absence of solvent(s), and

wherein the surface-reacted calcium carbonate of step a) and the one or more solid active(s) of step b) and optionally the additive(s) are mixed before co-grinding step c) is carried out.

It is appreciated that the mixing of the surface-reacted calcium carbonate of step a) and the one or more solid active(s) of step b), and if present the additive(s), may be carried out in any mixing device known to the skilled person. The skilled person also knows how to adapt mixing conditions such as mixing speed or mixing time.

Depending on the device used for co-grinding, the method may further comprise a step of separating the co-ground active(s) comprising product from grinding media such as grinding balls.

In this embodiment, the co-ground active(s) comprising product is preferably prepared by a method comprising, preferably consisting of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,
d) optionally contacting the surface-reacted calcium carbonate of step a) before, during or after co-grinding step c) with additive(s),
e) separating the co-ground active(s) comprising product obtained in step c), or optionally step d), from grinding media,

wherein the co-grinding in step c), and the contacting in optional step d), is/are carried out in the absence of solvent(s), and optionally the surface-reacted calcium carbonate of step a) and the one or more solid active(s) of step b) and optionally the additive(s) are mixed before co-grinding step c) is carried out.

According to a further aspect, the present invention relates to a method for preparing the co-ground active(s) comprising product, the method comprising, preferably consisting of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,

wherein the co-grinding in step c) is carried out in the absence of solvent(s).

With regard to the definition of the surface-reacted calcium carbonate, the active(s), the co-ground active(s) comprising product and preferred embodiments thereof, reference is made to the statements provided above when discussing the technical details of the co-ground active(s) comprising product of the present invention.

It is appreciated that the co-grinding in step c) can be carried out in any milling device known by the skilled person in methods for preparing the products to be prepared. In particular, the co-grinding in step c) is carried out in the absence of solvent(s) and thus a milling device is to be chosen suitable for dry grinding.

For example, the co-grinding in step c) is carried out in a mill, preferably selected from a ball mill, such as a planetary ball mill, roller mill, table mill, sand mill, ring roller mill, rod mill, vibrating mill, centrifugal impact mill, vertical bead mill and attrition mill. Most preferably, the co-grinding in step c) is carried out in a ball mill, such as a planetary ball mill.

It is to be noted that the skilled person is well aware of these devices and conditions to be applied for these devices. Thus, the skilled person is able to adjust the conditions for preparing the present co-ground active(s) comprising product, such as milling speed, milling time or the material and dimensions of possible milling balls, to the specific mill used.

In one embodiment, the co-grinding in step c) is carried out at room temperature. For example, the co-grinding in step c) is carried out at a milling starting temperature of below 60° C., preferably below 50° C. and most preferably below 40° C. For example, the co-grinding in step c) is carried out at a milling starting temperature in the range from 10 to 30° C. Alternatively, the co-grinding in step c) is carried out by cryo-milling. For example, the co-grinding in step c) is carried out at a milling starting temperature of about -196° C. However, it is to be noted that the given milling starting temperature does not exclude that the temperature rises above the milling starting temperature during the co-grinding and thus exceed the milling starting temperature.

In general, the co-grinding in step c) can be carried out for a few minutes to several hours for example from 1 min to 10 hours, depending on the devices used. For example, the co-grinding in step c) is carried out for 1 min to 60 min, preferably from 2 min to 40 min and most preferably for 5 min to 20 min, when using a ball mill, such as a planetary ball mill..

Additionally, the co-grinding in step c) can be carried out at a low energy input compared to other carrier materials such as silicates and calcium hydrogen phosphate. Preferably, the co-grinding in step c) is carried out at an energy input of at least 100 kJ/kg, preferably at least 150 kJ/kg, more preferably at least 200 kJ/kg and most preferably in the range from 200 to 1300 kJ/kg.

Depending on the devices used, the mill speed for the co-grinding in step c) can be adjusted to a mill speed of at least 100 rpm, preferably to a range from 100 to 2000 rpm. For example, the co-grinding in step c) is carried out at a mill speed of at least 200 rpm, preferably at least 300 rpm, more preferably at least 400 rpm and most preferably at least 450 rpm, when using a ball mill, such as as a planetary ball mill. Additionally or alternatively, the co-grinding in step c) is carried out at a mill speed of at most 1500 rpm, preferably at most 1000 rpm, more preferably at most 700 rpm and most preferably at most 600 rpm, when using a ball mill, such as a planetary ball mill. In one embodiment, the co-grinding in step c) is carried out at a mill speed ranging from 200 to 1500 rpm, preferably from 300 to 1000 rpm, more preferably from 400 to 700 rpm, and most preferably from 450 to 600 rpm when using a ball mill, such as a planetary ball mill.

In one embodiment, the milling balls used in the co-grinding in step c) have a size of 5 to 20 mm, preferably of 10 or 15 mm, when using a ball mill, such as a planetary ball mill.

The co-grinding in step c) results in a reduction of the volume median grain diameter d₅₀ of the surface-reacted calcium carbonate provided in step a). For example, the volume median grain diameter d₅₀ of the surface-reacted calcium carbonate provided in step a) is reduced in co-grinding step c) by more than 20%, preferably, more than 30% and most preferably more than 50%.

It is to be noted that the co-grinding in step c) results in a co-ground active(s) comprising product in which the one or more at least partially X-ray amorphous active(s) at least partially, preferably substantially completely, covers the surface of the carrier material being surface-reacted calcium carbonate.

The co-ground active(s) comprising product of the present invention and preferably obtained in step c), optionally obtained in step d) or step e), of the method is prepared in the absence of solvent(s). Thus, the co-ground active(s) comprising product of the present invention is preferably free of liquid materials and thus in a dry form. That is to say, the co-ground active(s) comprising product of the present invention is a solid material, i.e. a non-gaseous and non-liquid material, comprising or consisting of the surface-reacted calcium carbonate, the one or more active(s), and optionally additive(s).

In view of the above, it is appreciated that the co-ground active(s) comprising product is a product in which the one or more at least partially X-ray amorphous active(s) is/are closely associated to the surface of the carrier material being surface-reacted calcium carbonate. Thus, the co-ground active(s) comprising product of the present invention can be readily distinguished from a loose mixture comprising the same one or more active(s) and surface-reacted calcium carbonate.

In one embodiment, the co-ground active(s) comprising product comprises additive(s). In this case, the point at which the additive(s) is/are added in the method depends on the additive(s) used. Thus, the method further comprises a step d) of contacting the surface-reacted calcium carbonate of step a) before, during or after co-grinding step c) with additive(s).

Thus, if additive(s) are present, the method for preparing the co-ground active(s) comprising product comprises, preferably consists of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,
d) contacting the surface-reacted calcium carbonate of step a) before, during or after co-grinding step c) with additive(s),

wherein the co-grinding in step c) and the contacting in step d) are carried out in the absence of solvent(s).

If present, contacting step d) comprises a mixing of the surface-reacted calcium carbonate of step a) before, during or after co-grinding step c) and the additive(s).

In one embodiment, the surface-reacted calcium carbonate and the one or more solid active(s) may be mixed before co-grinding step c) is carried out. Thus, a pre-mixed mixture of the surface-reacted calcium carbonate and the one or more solid active(s) is subjected to co-grinding step c).

If such mixing step is present, the method for preparing the co-ground active(s) comprising product comprises, preferably consists of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,

wherein the co-grinding in step c) is carried out in the absence of solvent(s), and

wherein the surface-reacted calcium carbonate of step a) and the one or more solid active(s) of step b) are mixed before co-grinding step c) is carried out.

Furthermore, if additive(s) are added, the method for preparing the co-ground active(s) comprising product comprises, preferably consists of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,
d) contacting the surface-reacted calcium carbonate of step a) before, during or after co-grinding step c) with additive(s),

wherein the co-grinding in step c) and the contacting in step d) are carried out in the absence of solvent(s), and

wherein the surface-reacted calcium carbonate of step a) and the one or more solid active(s) of step b) and optionally the additive(s) are mixed before co-grinding step c) is carried out.

It is appreciated that the mixing of the surface-reacted calcium carbonate of step a) and the one or more solid active(s) of step b), and if present the additive(s), may be carried out in any mixing device known to the skilled person. The skilled person also knows how to adapt mixing conditions such as mixing speed or mixing time.

Depending on the device used for co-grinding step c), the method may further comprise a step of separating the co-ground active(s) comprising product from grinding media such as grinding balls.

In this embodiment, the method for preparing the co-ground active(s) comprising product comprises, preferably consists of, the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product,
d) optionally contacting the surface-reacted calcium carbonate of step a) before, during or after co-grinding step c) with additive(s),
e) separating the co-ground active(s) comprising product obtained in step c), or optionally step d), from grinding media,

wherein the co-grinding in step c), and the contacting in optional step d), is/are carried out in the absence of solvent(s), and optionally the surface-reacted calcium carbonate of step a) and the one or more solid active(s) of step b) and optionally the additive(s) are mixed before co-grinding step c) is carried out.

It is appreciated that the co-ground active(s) comprising product of the present invention can be used as such in any pharmaceutical, nutraceutical, veterinary or agricultural product.

Thus, the present invention refers in another aspect to a pharmaceutical, nutraceutical or agricultural product comprising the co-ground active(s) comprising product.

However, it is also possible that the co-ground active(s) comprising product is formulated with suitable excipients for the preparation of a pharmaceutical, nutraceutical, veterinary or agricultural product. Such excipients are well known in the art such that the skilled person will adapt the specific compositions according to the specific application and product to be prepared.

The pharmaceutical, nutraceutical or veterinary product can thus be in any form suitable for the intended application e.g. oral, dermatological, eye, nasal, intravenous, intramuscular, vaginal or rectal application. The agricultural product can be in any form typically used for the products to be prepared such as any kind of liquid or dry applications, e.g. spray or powder applications.

Preferably, the pharmaceutical, nutraceutical, veterinary or agricultural product is in the form of a liquid dosage form, preferably a liquid dosage form such as an emulsion, dispersion, creme, solution, spray or inhalation spray. Alternatively, the pharmaceutical, nutraceutical, veterinary or agricultural product is in the form of a solid dosage form, preferably a solid dosage form such as a powder, tablet, mini-tablet, sachet, granule, capsule, suppositories, or film.

In view of the favorable characteristics of the present co-ground active(s) comprising product, another aspect of the present invention relates to the use of the surface-reacted calcium carbonate as a carrier material for reducing the X-ray crystallinity of solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment.

With regard to the definition of the surface-reacted calcium carbonate, the active(s), the co-ground active(s) comprising product and preferred embodiments thereof, reference is made to the statements provided above when discussing the technical details of the co-ground active(s) comprising product of the present invention.

The following examples and tests will illustrate the present invention, but are not intended to limit the invention in any way.

### BRIEF DESCRIPTION OF THE FIGURES

FIG. 1 shows the porosimetry curves of SRCC1 as prepared and milled with or without ibuprofen.

FIG. 2 shows the porosimetry curves of SRCC1 as prepared and milled with or without piroxicam.

FIG. 3 shows the porosimetry curves of SRCC2 as prepared and milled with or without ibuprofen.

FIG. 4 refers to the % XRD crystallinity of the sample series 1, i.e. SRCC1 and SRCC2 as well as Emcompress® samples co-ground with 10 wt% ibuprofen.

FIG. 5 refers to the % XRD crystallinity of the sample series 1, i.e. SRCC1 and SRCC2 as well as Emcompress® samples co-ground with 10 wt% piroxicam.

FIG. 6 refers to the % XRD crystallinity of the sample series 1, i.e. SRCC1 and SRCC2 samples co-ground with 10 wt% dimethomorph.

FIG. 7 refers to the % XRD crystallinity of the sample series 2, i.e. SRCC1 and SRCC2 samples co-ground with 50 wt% dimethomorph.

FIG. 8 refers to the % XRD crystallinity of the sample series 1, i.e. SRCC1 and SRCC2 samples co-ground with 10 wt% sucrose.

FIG. 9 refers to the % XRD crystallinity of the sample series 2, i.e. SRCC1 and SRCC2 samples co-ground with 50 wt% sucrose.

### EXAMPLES

### 1. Measurement Methods

In the following, measurement methods implemented in the examples are described.

### Particle Size Distribution

Volume determined median particle size d₅₀(vol) and the volume determined top cut particle size d₉₈(vol) was evaluated using a Malvern Mastersizer 3000 Laser Diffraction System (Malvern Instruments Plc., Great Britain). The d₅₀(vol) or d₉₈(vol) value indicates a diameter value such that 50 % or 98 % by volume, respectively, of the particles have a diameter of less than this value. The raw data obtained by the measurement was analyzed using the Mie theory, with a particle refractive index of 1.57 and an absorption index of 0.005. The methods and instruments are known to the skilled person and are commonly used to determine particle size distributions of fillers and pigments. The sample was measured in dry condition without any prior treatment.

The weight determined median particle size d₅₀(wt) was measured by the sedimentation method, which is an analysis of sedimentation behaviour in a gravimetric field. The measurement was made with a Sedigraph™ 5120 of Micromeritics Instrument Corporation, USA. The method and the instrument are known to the skilled person and are commonly used to determine particle size distributions of fillers and pigments. The measurement was carried out in an aqueous solution of 0.1 wt.-% Na₄P₂O₇. The samples were dispersed using a high speed stirrer and supersonicated.

### Specific Surface Area (SSA)

The specific surface area was measured via the BET method according to ISO 9277:2010 using nitrogen. The samples were pre-dried in an oven at 200° C. for >4 h. The samples were then degassed in a VacPrep degassing unit for at least 60 min.

### Intra-Particle Intruded Specific Pore Volume (in Cm³/g)

The specific pore volume was measured using a mercury intrusion porosimetry measurement using a Micromeritics Autopore V 9620 mercury porosimeter having a maximum applied pressure of mercury 414 MPa (60000 psi), equivalent to a Laplace throat diameter of 0.004 µm. The equilibration time used at each pressure step was 20 seconds. The sample material was sealed in a 5 cm³ chamber powder penetrometer for analysis. The data were corrected for mercury compression, penetrometer expansion and sample material compression using the software Pore-Comp (Gane, P.A.C., Kettle, J.P., Matthews, G.P. and Ridgway, C.J., “Void Space Structure of Compressible Polymer Spheres and Consolidated Calcium Carbonate Paper-Coating Formulations”, Industrial and Engineering Chemistry Research, 35(5), 1996, p1753-1764.).

The total pore volume seen in the cumulative intrusion data can be separated into two regions with the intrusion data from 214 µm down to about 1 - 4 µm showing the coarse packing of the sample between any agglomerate structures contributing strongly. Below these diameters lies the fine inter-particle packing of the particles themselves. If they also have intra-particle pores, then this region appears bi-modal, and by taking the specific pore volume intruded by mercury into pores finer than the modal turning point, i.e. finer than the bi-modal point of inflection, the specific intra-particle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and inter-agglomerate pore volume from the total pore volume to deliver the desired pore volume of the internal pores alone in terms of the pore volume per unit mass (specific pore volume). The same principle of subtraction, of course, applies for isolating any of the other pore size regions of interest.

### SEM

A double faced adhesive tape (C-Tape, electrically conducting) was mounted on a sample holder. The powder sample was put on the tape and spread by tapping the sample holder while keeping the sample holder horizontal. A surplus of powder was removed by tapping the holder while holding it at an angle and carefully applying compressed CO2. The stub was then sputtered with 8 nm Au. The investigation under the FESEM (Zeiss Sigma VP) was done at 5kV using secondary electron detector (SE2).

### X-Ray Diffraction (XRD) Analysis

The mineralogical composition of the prepared samples were analysed by means of X-ray diffraction (XRD) obeying Bragg’s law, using either of two systems: System 1: a Bruker D8 Advance powder diffractometer with a 2.2 kW X-ray tube, a sample holder, a ν-ν goniometer, and a VÅNTEC-1 detector, scanning at 0.7 ° per minute in 2ν; or system 2: A Bruker D8 Advance ECO powder diffractometer with a 1 kW X-ray tube, a sample holder, a ν-ν goniometer, and a LYNXEYE XE-T detector, scanning at 0.02 ° per second in 2ν. Nickel-filtered Cu Kα radiation was employed in all experiments. The resulting powder diffraction pattern was interpreted with respect to mineral content using the Bruker DIFFRACsuite software package EVA in comparison to the ICDD PDF 2 database (XRD LTM 7603) of reference patterns.

The following samples were measured with system 1: Samples 1 c, d, f.

The remaining samples were measured with system 2.

The ratio of hydroxyapatite to calcium carbonate for the surface-reacted calcium carbonate was measured by using system 1.

Quantitative analysis of the diffraction data, i.e., the determination of amounts of different phases in a multi-phase sample, has been performed using the Bruker DIFFRACsuite software package TOPAS. This involved iterative modelling of the full diffraction pattern (Rietveld refinement), minimizing the differences between modelled and measured patterns. The Rietveld method requires knowledge of the approximate crystal structure of all phases of interest in the pattern. However, the use of the whole pattern rather than a few select lines produces accuracy and precision much better than any single-peak-intensity based method.

Semi-Quantitative (SQ) calculations to estimate the rough mineral concentrations were carried out with the DIFFRAC^{suite} software package EVA. The semi-quantitative analysis was performed considering the patterns relative heights and I/I_{cor} values (I/I_{cor}: ratio between the intensities of the strongest line in the compound of interest and the strongest line of corundum, both measured from a scan made of a 50-50 by weight mixture).

Crystallite size can be determined from the peak-broadening of the respective phase. The Rietveld refinement also included modelling the peak shape based on a Lorentzian-type component convolution. The integral breadth (IB) of such modelled peaks was then used to estimate the crystallite size (LVol-IB), largely independent of crystallite shape.

### 2. Materials Used

### Surface-Reacted Calcium Carbonates

### SRCC1

Surface-reacted calcium carbonate (SRCC1) (d₅₀(vol) = 6.6 µm, d₉₈(vol) = 13.7 µm, SSA = 59.9 m²/g). The intra-particle intruded specific pore volume is 0.939 cm³/g (for the pore diameter range of 0.004 to 0.51 µm). SRCC1 provides a ratio of hydroxyapatite to calcium carbonate of about 50:50 as determined by XRD.

SRCC1 was obtained by preparing 350 litres of an aqueous suspension of ground calcium carbonate in a mixing vessel by adjusting the solids content of a ground limestone calcium carbonate from Omya SAS, Orgon having a weight based median particle size d₅₀(wt) of 1.3 µm, as determined by sedimentation, such that a solids content of 10 wt.-%, based on the total weight of the aqueous suspension, is obtained.

Whilst mixing the slurry at a speed of 6.2 m/s, 11.2 kg phosphoric acid was added in form of an aqueous solution containing 30 wt.-% phosphoric acid to said suspension over a period of 20 minutes at a temperature of 70° C. After the addition of the acid, the slurry was stirred for additional 5 minutes, before removing it from the vessel and drying using a jet-dryer.

### SRCC2

SRCC 2 had a d₅₀(vol) = 7.8 µm, d₉₈(vol) = 20 µm, SSA = 81 m²/g with an intra-particle intruded specific pore volume of 1.218 cm³/g (for the pore diameter range of 0.004 to 0.38 µm). SRCC2 provides a ratio of hydroxyapatite to calcium carbonate of about 85:15 as determined by XRD.

SRCC2 was obtained by preparing 2000 litres of an aqueous suspension of ground calcium carbonate in a mixing vessel by adjusting the solids content of a ground limestone calcium carbonate from Orgon, France having a mass based particle size distribution of 80% less than 1 µm, as determined by sedimentation, such that a solids content of 13 wt%, based on the total weight of the aqueous suspension, is obtained.

Whilst mixing the slurry, 715 kg of an aqueous phosphoric acid solution was added over 15 minutes, wherein said solution contained 20 wt% phosphoric acid. The temperature of the suspension was maintained at 70° C. After the addition of the acid, the suspension was stirred for a minimum of 5 minutes before removing it from the vessel and drying.

### Other Materials

Emcompress® Anhydrous Powder; Calcium Hydrogen Phosphate from JRS Pharma GmbH, having a d₅₀(vol) of 13.65 µm, d₉₈(vol) of 48 µm, BET specific surface area of 3.6 m²/g

Ibuprofen from Shasun Pharmaceuticals Limited

Piroxicam from Ramdev Chemical PVT. LTD

### Preparation of Milled Compounds

### Using a Planetary Ball Mill (Retsch PM 100)

### Preparation of Samples 1 With 10% Active

3.60 g of carrier material and 0.4 g of active were combined in a 50 ml Wolfram carbid (WC) vessel and stirred with a spatula to combine. 1010 mm WC balls or 715 mm WC balls were carefully added to the vessel. The vessel was then closed with the respective lid and mounted in the milling device. The milling time was set to 10 min with 1 min intervals and reversal of rotation direction every minute. The mill speed was set to 300, 400, 500 or 550 rpm. After the milling time, the vessel was removed from the milling device and the milling balls were removed using a 0.5 mm sieve. The resulting compounds were collected and analysed.

### Preparation of Samples 2 With 50% Active

2 g of carrier material and 2 g of active were combined in a 50 ml Wolfram carbid (WC) vessel and stirred with a spatula to combine. 1010 mm WC balls or 715 mm WC balls were carefully added to the vessel. The vessel was then closed with the respective lid and mounted in the milling device. The milling time was set to 10 min with 1 min intervals and reversal of rotation direction every minute. The mill speed was set to 550 rpm. After the milling time, the vessel was removed from the milling device and the milling balls were removed using a 0.5 mm sieve. The resulting compounds were collected and analysed.

The results for samples 1 and 2 are set out in the following table 1.

TABLE 1

Samples prepared using the planetary ball mill Retsch PM100

| Sample | Carrier | Active | Amount Carrier [g] | Amount Active [g] | Total Milling Mass [g] | Loading [%] | Ball size [mm] | Planetary Mill Speed [rpm] | Milling time [min] | XRD crystallinity of Active [%] |
|---|---|---|---|---|---|---|---|---|---|---|
| Sample 1 a | SRCC1 | Ibuprofen | 3.6 | 0.4 | 4 | 10% | 10 | 500 | 10 | 20% |
| Sample 1 b | SRCC1 | Ibuprofen | 3.6 | 0.4 | 4 | 10% | 15 | 500 | 10 | 10% |
| Sample 1 c | SRCC1 | Ibuprofen | 3.6 | 0.4 | 4 | 10% | 10 | 550 | 10 | 1% |
| Sample 1 d | SRCC1 | Ibuprofen | 3.6 | 0.4 | 4 | 10% | 15 | 550 | 10 | 1% |
| Sample 1 e | SRCC2 | Ibuprofen | 3.6 | 0.4 | 4 | 10% | 10 | 300 | 10 | 10% |
| Sample 1 f | SRCC2 | Ibuprofen | 3.6 | 0.4 | 4 | 10% | 10 | 400 | 10 | 1% |
| Sample 1 g | SRCC2 | Ibuprofen | 3.6 | 0.4 | 4 | 10% | 10 | 550 | 10 | 0% |
| Sample 1 h | Emcompress | Ibuprofen | 3.6 | 0.4 | 4 | 10% | 10 | 550 | 10 | 23% |
| Sample 1 i | Emcompress | Ibuprofen | 3.6 | 0.4 | 4 | 10% | 15 | 550 | 10 | 15% |
| Sample 1 j | SRCC1 | Piroxicam | 3.6 | 0.4 | 4 | 10% | 10 | 550 | 10 | 39% |
| Sample 1 k | SRCC1 | Piroxicam | 3.6 | 0.4 | 4 | 10% | 15 | 550 | 10 | 1% |
| Sample 1 l | SRCC2 | Piroxicam | 3.6 | 0.4 | 4 | 10% | 15 | 550 | 10 | 1% |
| Sample 1 m | SRCC2 | Piroxicam | 3.6 | 0.4 | 4 | 10% | 10 | 550 | 10 | 0% |
| Sample 1 n | Emcompress | Piroxicam | 3.6 | 0.4 | 4 | 10% | 15 | 550 | 10 | 33% |
| Sample 1 o | SRCC1 | Dimethomorph | 3.6 | 0.4 | 4 | 10% | 10 | 550 | 10 | 0% |
| Sample 1 p | SRCC1 | Dimethomorph | 3.6 | 0.4 | 4 | 10% | 15 | 550 | 10 | 0% |
| Sample 1 q | SRCC2 | Dimethomorph | 3.6 | 0.4 | 4 | 10% | 10 | 550 | 10 | 0% |
| Sample 1 r | SRCC2 | Dimethomorph | 3.6 | 0.4 | 4 | 10% | 15 | 550 | 10 | 0% |
| Sample 1 s | SRCC1 | Sucrose | 3.6 | 0.4 | 4 | 10% | 10 | 550 | 10 | 0% |
| Sample 1 t | SRCC1 | Sucrose | 3.6 | 0.4 | 4 | 10% | 15 | 550 | 10 | 0% |
| Sample 1 u | SRCC2 | Sucrose | 3.6 | 0.4 | 4 | 10% | 10 | 550 | 10 | 0% |
| Sample 1 v | SRCC2 | Sucrose | 3.6 | 0.4 | 4 | 10% | 15 | 550 | 10 | 0% |
| Sample 2 a | SRCC1 | Dimethomorph | 2 | 2 | 4 | 50% | 10 | 550 | 10 | 40% |
| Sample 2 b | SRCC1 | Dimethomorph | 2 | 2 | 4 | 50% | 15 | 550 | 10 | 42% |
| Sample 2 c | SRCC2 | Dimethomorph | 2 | 2 | 4 | 50% | 10 | 550 | 10 | 42% |
| Sample 2 d | SRCC2 | Dimethomorph | 2 | 2 | 4 | 50% | 15 | 550 | 10 | 38% |
| Sample 2 e | SRCC1 | Sucrose | 2 | 2 | 4 | 50% | 10 | 550 | 10 | 28% |
| Sample 2 f | SRCC1 | Sucrose | 2 | 2 | 4 | 50% | 15 | 550 | 10 | 30% |
| Sample 2 g | SRCC2 | Sucrose | 2 | 2 | 4 | 50% | 10 | 550 | 10 | 36% |
| Sample 2 h | SRCC2 | Sucrose | 2 | 2 | 4 | 50% | 15 | 550 | 10 | 38% |

From table 1, it can be gathered that increasing the ball size or the rpm (mill speed) increases the energy delivered into the system. Therefore milling the same product at higher rpm results in more X-ray amorphous, i.e. less crystalline actives. The same applies for increasing the ball size. To evaluate milling efficiency with different carriers, only experiments with the same composition, rpm, and balls size are to be compared.

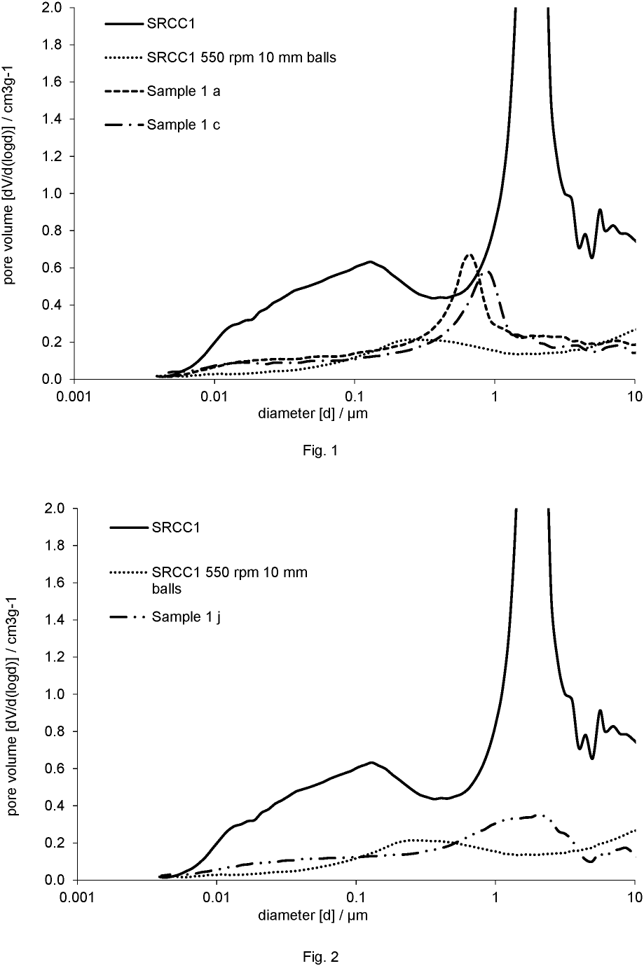

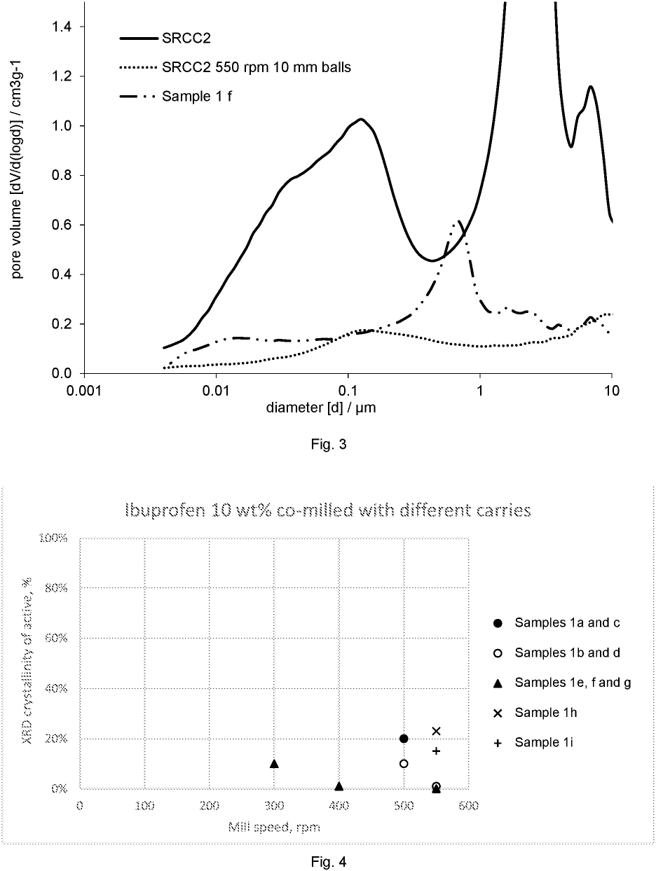

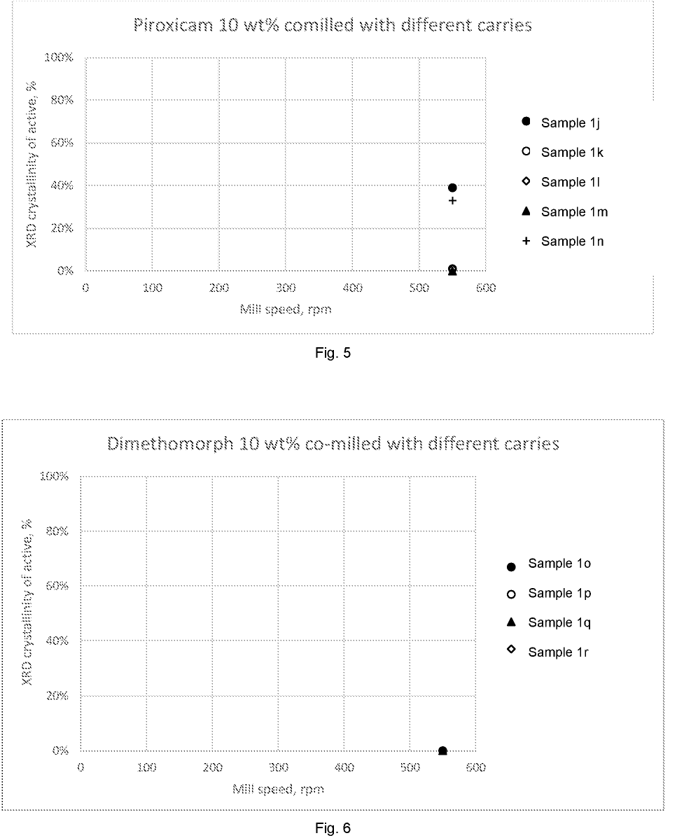

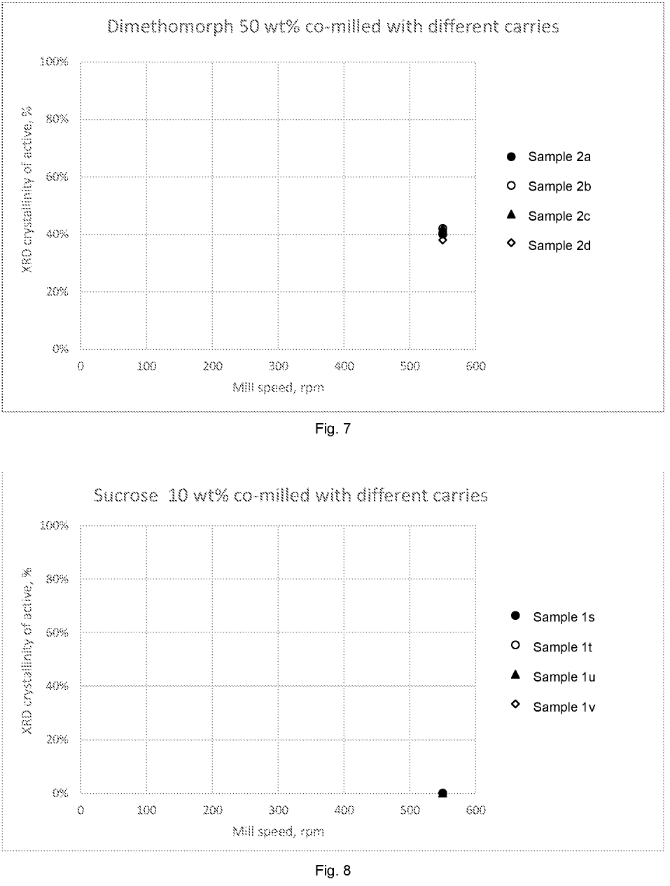

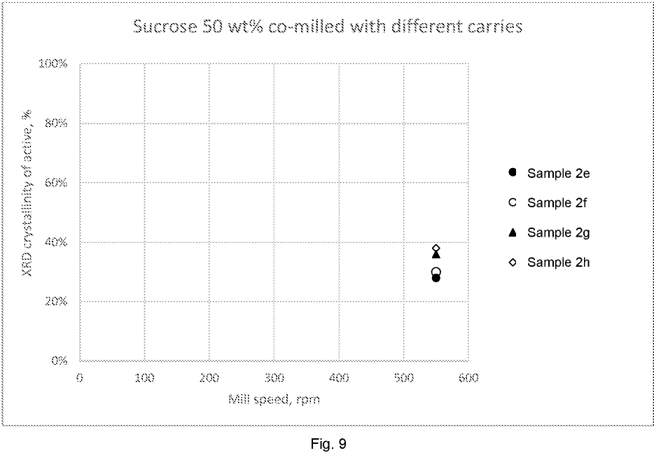

Porosimetry curves of SRCC1 samples as prepared and milled with or without ibuprofen are shown in FIG. 1. It can be gathered that the intra-particle pore space (0.004 to 0.5 µm) as well as the interparticle space (0.5 to 10 µm) is reduced upon milling. Furthermore, porosimetry curves of SRCC1 as prepared and milled with or without piroxicam are shown in FIG. 2. The intra-particle pore space (0.004 to 0.5 µm) as well as the interparticle space (0.5 to 10 µm) is also reduced upon milling. Porosimetry curves of SRCC2 as prepared and milled with or without ibuprofen are shown in FIG. 3. The intra-particle pore space (0.004 to 0.5 µm) as well as the interparticle space (0.5 to 10 µm) is reduced upon milling. The % XRD crystallinity of the sample series 1, i.e. SRCC1 and SRCC2 as well as Emcompress® samples co-ground with 10 wt% ibuprofen as set out in Table 1, is shown in FIG. 4. It is to be noted that the samples are prepared by using different ball sizes (10 and 15 mm, respectively) and different milling speeds (300, 400, 500 and 550 rpm, respectively). The % XRD crystallinity of the sample series 1, i.e. SRCC1 and SRCC2 as well as Emcompress® samples co-ground with 10 wt% piroxicam as set out in Table 1, is shown in FIG. 5. It is to be noted that the samples are prepared by using different ball sizes (10 and 15 mm, respectively) at a milling speed of 550 rpm. The % XRD crystallinity of the sample series 1, i.e. SRCC1 and SRCC2 samples co-ground with 10 wt% dimethomorph as set out in Table 1, is shown in FIG. 6. It is to be noted that the samples are prepared by using different ball sizes (10 and 15 mm, respectively) at a milling speed of 550 rpm. The % XRD crystallinity of the sample series 2, i.e. SRCC1 and SRCC2 samples co-ground with 50 wt% dimethomorph as set out in Table 1, is shown in FIG. 7. It is to be noted that the samples are prepared by using different ball sizes (10 and 15 mm, respectively) at a milling speed of 550 rpm. The % XRD crystallinity of the sample series 1, i.e. SRCC1 and SRCC2 samples co-ground with 10 wt% sucrose, as set out in Table 1, is shown in FIG. 8. It is to be noted that the samples are prepared by using different ball sizes (10 and 15 mm, respectively) at a milling speed of 550 rpm. The % XRD crystallinity of the sample series 2, i.e. SRCC1 and SRCC2 samples co-ground with 50 wt% sucrose as set out in Table 1, is shown in FIG. 9. It is to be noted that the samples are prepared by using different ball sizes (10 and 15 mm, respectively) at a milling speed of 550 rpm.

### Comparative Example 1

Comparative example 1 was prepared according to the information given in EP3260114 A1, i.e. by loading surface-reacted calcium carbonate with at least one active ingredient and/or inactive precursor thereof, compacting the loaded surface-reacted calcium carbonate obtained by means of a roller compacter at a compaction pressure in the range from 1 to 30 kN/cm into a compacted form; and milling the compacted form into granules.

In particular, 300 g of SRCC1 were placed on a 3 L plastic beaker. The powder was loaded with 33.4 g (10 wt.-%) of ibuprofen. The ibuprofen was first dissolved in 150 g acetone. The ibuprofen acetone solution was loaded by spraying at a rate of 5 hits every 15 seconds by means of a spray bottle. While leading, the powder was permanently mixed with an overhead stirrer IKA RW2O at a speed ranging between 80 and 120 rpm using an open blade paddle mixer. After the total amount of solution was loaded onto the FCC, the loaded powder was left to mix 10 minutes longer. The loaded powder was dried at a vacuum oven ThermoScientific VT 6130 until no more solvent could be collected. The granulation was performed using the Fitzpatrick CCS220. A bar mill and a rasped 1 mm screen with bar rotor were used for granulation. The parameter settings for granulation are set out in the following table 2.

TABLE 2

parameter settings for comparative example 1

| | |
|---|---|
| Roll gap | 0.7 mm (actual value during process 0.8 rpm) |
| Roll force | 3 kN/cm |
| Roll speed | 7 rpm |
| Horizontal screw speed | 25 rpm |
| Vertical screw speed | 250 rpm |
| Mill speed | 300 rpm |

The granule fraction between 250-710 µm was produced using a Retsch tower sieve shaker A3300 with 90, 180, 250, 355, 500, 710 and 1 000 µm.

Comparative example 1 has a crystallinity of 50 wt.-%, based on the total weight of the active.

### Comparative Example 2

Comparative example 2 was prepared according to the information given in EP3260114 A1, i.e. by loading surface-reacted calcium carbonate with at least one active ingredient and/or inactive precursor thereof, compacting the loaded surface-reacted calcium carbonate obtained by means of a roller compacter at a compaction pressure in the range from 1 to 30 kN/cm into a compacted form; and milling the compacted form into granules.

In particular, 300 g of SRCC1 were placed on a 3 L plastic beaker. The powder was loaded with 33.4 g (10 wt.-%) of ibuprofen. The ibuprofen was first dissolved in 150 g acetone. The ibuprofen acetone solution was loaded by spraying at a rate of 5 hits every 15 seconds by means of a spray bottle. While leading, the powder was permanently mixed with an overhead stirrer IKA RW2O at a speed ranging between 80 and 120 rpm using an open blade paddle mixer. After the total amount of solution was loaded onto the FCC, the loaded powder was left to mix 10 minutes longer. The loaded powder was dried at a vacuum even ThermoScientific VT 6130 until no more solvent could be collected. The granulation was performed using the Fitzpatrick CCS220. A bar mill and a rasped 1 mm screen with bar rotor were used for granulation. The parameter settings for granulation are set out in the following table 3.

TABLE 3

parameter settings for comparative example 2

| | |
|---|---|
| Roll gap | 0.7 mm (actual value during process 0.8 rpm) |
| Roll force | 3 kN/cm |
| Roll speed | 3 rpm |
| Horizontal screw speed | 8 rpm |
| Vertical screw speed | 250 rpm |
| Mill speed | 300 rpm |

The granule fraction between 250-710 µm was produced using a Retsch tower sieve shaker A3300 with 90, 180, 250, 355, 500, 710 and 1000 µm.

Comparative example 2 has a crystallinity of 50 wt.-%, based on the total weight of the active.

1. Co-ground active(s) comprising product comprising a carrier material being surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donors treatment, and one or more at least partially X-ray amorphous active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof, wherein the one or more active(s) is/are rendered at least partially X-ray amorphous during co-grinding.

2. The co-ground active(s) comprising product according to claim 1, wherein the one or more at least partially X-ray amorphous active(s) has/have a crystallinity of less than 50 wt.-%, preferably of less than 40 wt.-%, more preferably of less than 30 wt.-% and most preferably of less than 20 wt.-%, based on the total weight of the one or more at least partially X-ray amorphous active.

3. The co-ground active(s) comprising product according to claim 1 , wherein the natural ground calcium carbonate is selected from calcium carbonate containing minerals selected from the group comprising marble, chalk, limestone and mixtures thereof.

4. The co-ground active(s) comprising product according to claim 1, wherein the surface-reacted calcium carbonate is a reaction product of natural ground calcium carbonate with carbon dioxide and phosphoric acid, wherein the carbon dioxide is formed in-situ by the phosphoric acid treatment.

5. The co-ground active(s) comprising product according to claim 1, wherein the one or more at least partially X-ray amorphous active(s) has/have a melting point of at least 30° C., more preferably at least 35° C. and most preferably in the range from 35 to 400° C.

6. The co-ground active(s) comprising product according to claim 1, wherein the co-ground active(s) product comprises the one or more at least partially X-ray amorphous active(s) in an amount ranging from 1 to 45 wt.-%, preferably from 2 to 35 wt.-% and most preferably from 3 to 30 wt.-%, based on the total weight of the co-ground active(s) comprising product.

7. The co-ground active(s) comprising product according to claim 1, wherein the carrier material of the co-ground active(s) comprising product is free of materials differing from surface-reacted calcium carbonate.

8. The co-ground active(s) comprising product according to claim 1, wherein the co-ground active(s) comprising product is prepared according to a method comprising the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product, wherein the co-grinding in step c) is carried out in the absence of solvent(s) .

9. Method for preparing a co-ground active(s) comprising product according to claim 1, the method comprising the steps of:
a) providing a surface-reacted calcium carbonate, wherein the surface-reacted calcium carbonate is a reaction product of natural ground calcium carbonate with carbon dioxide and one or more H₃O⁺ ion donors, wherein the carbon dioxide is formed in-situ by the H₃O⁺ ion donorstreatment,
b) providing one or more solid active(s) selected from the group comprising pharmaceutical active(s) and/or inactive precursor thereof, nutraceutical active(s) and/or inactive precursor thereof, veterinary active(s) and/or inactive precursor thereof, agricultural active(s) and/or inactive precursor thereof, and mixtures thereof,
c) co-grinding the surface-reacted calcium carbonate of step a) and the one or more solid active(s) of step b) such as to obtain the co-ground active(s) comprising product, wherein the co-grinding in step c) is carried out in the absence of solvent(s).

10. The method according to claim 9, wherein the surface-reacted calcium carbonate provided in step a)
a) has a BET specific surface area of from 1 m²/g to 200 m²/g, preferably 2 m²/g to 150 m²/g, more preferably 20 m²/g to 140 m²/g, most preferably 40 m²/g to 110 m²/g, measured using nitrogen and the BET method according to ISO 9277:2010; and/or
b) comprises particles having a volume median grain diameter dso(vol) of from 0.5 to 50 µm, preferably from 0.7 to 25 µm, more preferably 0.8 to 20 µm, particularly 1 to 10 µm as measured by laser diffraction; and/or
c) has an intra-particle intruded specific pore volume within the range of 0.15 to 1.60 cm³/g, preferably from 0.30 to 1.50 cm³/g, more preferably from 0.30 to 1.40 cm³/g, and most preferably from 0.30 to 1.35 cm³/g, calculated from a mercury intrusion porosimetry measurement.

11. The method according to claim 9, wherein the co-grinding in step c) is carried out in a mill, preferably selected from a ball mill, such as a planetary ball mill, roller mill, table mill, sand mill, ring roller mill, rod mill, vibrating mill, centrifugal impact mill, vertical bead mill and attrition mill.

12. The method according to claim 9, wherein the volume median grain diameter d₅₀ of the surface-reacted calcium carbonate provided in step a) is reduced in co-grinding step c) by more than 20%.

13. The method according to claim 9, wherein the co-grinding in step c) is carried out at an energy input of at least 100 kJ/kg, preferably at least 150 kJ/kg, more preferably at least 200 kJ/kg and most preferably in the range from 200 to 1300 kJ/kg.

14. Pharmaceutical, nutraceutical, veterinary or agricultural product comprising the co-ground active(s) comprising product according to claim 1.

15. The pharmaceutical, nutraceutical, veterinary or agricultural product according to claim 14 in the form of a liquid dosage form, preferably a liquid dosage form such as an emulsion, dispersion, creme, solution, spray or inhalation spray, or solid dosage form, preferably a solid dosage form such as a powder, tablet, mini-tablet, sachet, granule, capsule, suppositories, or film.

16. A method of reducing the X-ray crystallinity of solid active(s), comprising co-grinding a surface-reacted calcium carbonate as a carrier material and one or more at least partially X-ray amorphous active(s),
wherein said one or more at least partially X-ray amorphous active(s) is selected from the group comprising pharmaceutical active(s) and/or inactive precursor thereof, veterinary active(s) and/or inactive precursor thereof, nutraceutical active(s) and/or inactive precursor thereof, agricultural active(s) and/or inactive precursor thereof, and mixtures thereof,
wherein the surface-reacted calcium carbonate is a reaction product of natural ground calcium carbonate with carbon dioxide and one or more H₃O⁺ ion donors, and wherein the carbon dioxide is formed in-situ by H₃O⁺ ion donors treatment.
